(19) United States
(12) Patent Application Publication
(10) Pub. No.: US 2007/0092949 A1
(43) Pub. Date: Apr. 26, 2007

(54) Method of converting beta-1,4-glucan to alpha-glucan

(76) Inventors: Koji Odan, Daito-shi (JP); Takeshi Takaha, Kobe-shi (JP); Takashi Kuriki, Suita-shi (JP); Kenichi Kudo, Yamatotakada-shi (JP); Mamoru Wada, Kitakatsuragi-gun (JP); Michihiro Sunako, Uda-gun (JP); Junichi Takahara, Kashiba-shi (JP)

Correspondence Address: MARK D. SARALINO (GENERAL);RENNER, OTTO, BOISSELLE & SKLAR, LLP, 1621 EUCLID AVENUE, NINETEENTH FLOOR, CLEVELAND, OH, 44115-2191, US

(21) Appl. No.: 10/596,243

(22) PCT Filed: Dec. 9, 2004
(86) PCT No.: PCT/JP04/18416

§ 371 Date: Nov. 17, 2006

(30) Foreign Application Priority Data
Dec. 12, 2003 (JP) 2003-415808

(51) Int. Cl. C12P 19/04

(52) U.S. Cl. 435/101; 536/123.12

(57) ABSTRACT

A method for production of an α-glucan from a β-1,4-glucan is provided, comprising: reacting a solution containing β-1,4-glucan, a primer, a source of phosphoric acid, β-1,4-glucan phosphorylase, and α-1,4-glucan phosphorylase to produce an α-glucan. In this method, said β-1,4-glucan may be cellobiose, and said β-1,4-glucan phosphorylase may be cellobiose phosphorylase.

### TECHNICAL FIELD

The present invention relates to a method for production of an α-glucan from a β-1,4-glucan.

### BACKGROUND ART

Human beings utilize α-glucans such as starch as an energy source upon digestion. α-glucan is widely utilized as a raw material in drugs, cosmetics, chemical industries, paper making, fibers and the like in addition to its use as a raw material in food industries, and is a very highly useful substance. Among α-glucans, particularly, amylose is expected to be utilized in a wide range of fields because it has a variety of functions.

In recent years, a food crisis has been regarded as a problem due to population increase, and it is expected that an energy source derived only from starches produced by plants will be deficient in the future.

On the other hand, since human beings cannot digest β-glucans such as cellulose, human beings cannot utilize β-glucans as an energy source, and β-glucans are utilized only as a food fiber component. Therefore, β-glucans cannot be utilized for solving a food crisis problem. However, the amount of β-glucans produced in one year is estimated to be about twenty thousand-fold the amount of starch, and there is no fear of exhaustion. For this reason, various attempts have been made to convert β-glucans into a substance which can be used as an energy source by human beings.

For example, study has been done to degrade cellulose to glucose and utilize this in ethanol fermentation. Glucose can be metabolized by human beings, but since it is too sweet, it cannot be ingested at large amounts as an energy source.

If β-glucans could be converted into a substance (particularly starch, which is also a polymer of glucose) which can be more easily ingested by human beings, this could greatly contribute to a solution of the food crisis problem, but such technology has not previously been disclosed.

Thus, the present inventors tried to produce an α-glucan using a β-glucan as a raw material. β-glucans cannot be converted directly into an α-glucan. In a previous method, a method for synthesizing a cellobiose from G-1-P and glucose by the action of cellobiose phosphorylase (CBP) is known. A method for synthesizing a cellooligosaccharide having a degree of polymerization of n+1 from G-1-P and a cellooligosaccharide (degree of polymerization n) by the action of cellodextrin phosphorylase (CDP) is also known. In addition, a method for synthesizing a high-molecular-weight α-glucan from G-1-P and a low-molecular-weight α-glucan by the action of α-1, 4-glucan phosphorylase is known. Generally, since a reaction which is catalyzed by an enzyme is a reversible reaction in many cases, the present inventors thought that a reaction catalyzed by CBP would proceed in the direction of cellobiose degradation, and a reaction catalyzed by CDP would proceed in the direction of cellooligosaccharide degradation to produce G-1-P, and an α-glucan can be synthesized from the resultant G-1-P. Then, the present inventors studied construction of a method for synthesizing an α-glucan from a β-1,4-glucan. This method is a two-step method in which β-glucan is phosphorolyzed using cellobiose phosphorylase (CBP) or cellodextrin phosphorylase (CDP) to obtain G-1-P (first step), and α-glucan is synthesized by glucan phosphorylase (GP) using this G-1-P as a raw material (second step). In the reaction of phosphorolyzing β-glucan in this method, in order to obtain G-1-P effectively, it is necessary to add a large amount of inorganic phosphoric acid. However, since this large amount of inorganic phosphoric acid inhibits the reaction of synthesizing α-glucan using G-1-P as a raw material, which is the next reaction, this inorganic phosphoric acid must be removed after completion of the reaction of the first step. However, this purification step is costly, and this is one drawback of this two-step method.

In addition, when one tries to synthesize α-glucan using G-1-P as a raw material, since an equimolar phosphoric acid is produced as a byproduct of the reaction, it becomes necessary to remove the byproduct phosphoric acid after completion of the reaction. In addition, since considerable reduction in pH due to the byproduct phosphoric acid is seen, a procedure of maintaining the pH of the reaction solution by adding an alkali and the like or using a high concentration buffer and the like becomes necessary. For these reasons, this two-step method cannot be said to be a simple production method.

For these reasons, development of a low cost, simple and efficient method which overcomes these drawbacks is desired.

A method of performing a reaction in a single reaction system by coupling respective enzymes in order to perform a catalytic reaction consisting of two enzymatic reaction steps has been developed in other catalytic reactions. The previously known example of such a reaction system is a method of utilizing two kinds of phosphorylases by coupling them. For example, Kitaoka et al. (Non-Patent Document 1) discloses the technology of converting sucrose into cellobiose efficiently by simultaneous action of sucrose phosphorylase (SP) and CBP. In addition, Fujii et al. (Patent Document 1) discloses the technology of converting sucrose into amylose efficiently by simultaneous action of SP and GP.

These technologies utilize a complicated reaction in which two kinds of enzymes share the substrate and the product (in an example of Kitaoka et al., G-1-P is a product of SP and, at the same time, a substrate for CBP, and phosphoric acid is a substrate for SP and, at the same time, a product of CBP). For this reason, the reaction mechanism is extremely complicated, and different from a reaction using a single enzyme. Furthermore, it is technical common knowledge that even when two kinds of enzymes are simply combined, a substrate as a raw material cannot be necessarily converted into an objective product.

At the Annual Meeting of The Japanese Society of Applied Glycoscience held in 2001, Kitaoka et al. orally reported that a system using SP and CBP can be effectively utilized in a method of synthesizing cellobiose from sucrose via G-1-P, but the reaction synthesizing sucrose from cellobiose via G-1-P does not proceed. The present inventors have performed a confirmation experiment (Reference Example 2) based on this report to confirm that the reaction synthesizing sucrose from cellobiose via G-1-P does not proceed.

That is, the enzymatic reaction for synthesizing G-1-P with CBP, and a further enzymatic reaction for synthesized G-1-P cannot be performed at the same time.

Therefore, it was thought to be difficult to perform a simultaneous two-step enzymatic reaction using cellobiose as a starting material.

Furthermore, other than a method proceeding via G-1-P, there was not a method for efficiently synthesizing an α-glucan from a β-1,4-glucan. Consequently, there was no low-cost, simple and efficient method synthesizing an α-glucan from a β-1,4-glucan.

Glucose is involved in the enzymatic reaction of producing an α-glucan from a β-1,4-glucan. For this reason, it can also be thought that by controlling the concentration of glucose it becomes possible to perform the desired enzymatic reaction efficiently.

Kitaoka et al. (Non-Patent Document 2) asserts that, in order to proceed the reaction towards cellobiose synthesis in a system synthesizing cellobiose from sucrose, it is important to maintain a low concentration of glucose, which is an essential raw material as an acceptor, in the reaction system. For this reason, by converting fructose produced by the action of SP into glucose using xylose isomerase, the reaction is made to proceed without adding glucose from outside of the system, thereby, the yield of cellobiose is increased. Kitaoka et al. explain that this is because accumulation of glucose at a high concentration remarkably reduces the reaction of synthesizing cellobiose by CBP, as glucose is an antagonistic inhibitor of CBP for G-1-P. Therefore, previously, it was thought that, in an enzymatic reaction using cellobiose as a substrate, it is important to reduce the concentration of glucose in the reaction solution in order to proceed the reaction towards cellobiose synthesis and, conversely, increase the concentration of glucose in the reaction solution in order to proceed the reaction in the direction of degradation of cellobiose.

The present invention includes a reaction using CBP to degrade cellobiose, which is a substrate therefor. Based on the aforementioned finding, those skilled in the art think that a high concentration of glucose is an advantageous condition for cellobiose degradation by inhibiting the cellobiose synthesizing reaction.

On the other hand, since in a two-enzyme reaction for converting cellobiose into amylose equilibrium of the reaction of CBP is controlled by the G-1-P/phosphoric acid ratio, and the glucose/CBP ratio, even when only the concentration of glucose is lowered, whether or not the whole reaction becomes advantageous for converting cellobiose into amylose is not clear. Actually, in a system for synthesizing sucrose from cellobiose, the reaction yield could not be increased even when the concentration of glucose is reduced (Reference Example 2). This means that, in a complicated reaction system combining two kinds of phosphorylase, the reaction yield is not improved even when the byproduct is eliminated.
Patent Document 1: International Publication No. 02/097107 Pamphlet
Non-Patent Document 1: Kitaoka et al., Denpun Kagaku, vol. 39, No. 4, 1992, pp. 281-283
Non-Patent Document 2: Kitaoka et al., Trends in Glycoscience and Glycotechnology, vol. 14, No. 75, 2002, pp. 35-50

### DISCLOSURE OF THE INVENTION

### PROBLEMS TO BE SOLVED BY THE INVENTION

The present invention intends to solve the aforementioned problems. An object of the present invention is to provide a method of efficiently converting a β-1,4-glucan, which cannot be food, into an α-glucan without proceeding via a complicated producing step.

### MEANS FOR SOLVING THE PROBLEMS

As a result of diligent study to solve the aforementioned problems, the present inventors found that, by coupling a reaction of phosphorolyzing a β-glucan in the presence of β-1,4-glucan phosphorylase to synthesize glucose-1-phosphate with a reaction of reacting glucose-1-phosphate with a primer in the presence of α-glucan phosphorylase to synthesize an α-glucan, an α-glucan is efficiently synthesized from a β-1,4-glucan. Based on these findings, the present inventors completed the present invention.

Also, the present inventors unexpectedly found that, contrary to the previous finding, in this reaction system, by decreasing the concentration of glucose produced when β-glucan is phosphorolyzed in the presence of β-1,4-glucan phosphorylase, α-glucan can be produced more efficiently.

The method of the present invention is a production method for an α-glucan from a β-1,4-glucan, and comprises reacting a solution containing a β-1,4-glucan, a primer, a source of phosphoric acid, β-1,4-glucan phosphorylase, and α-1,4-glucan phosphorylase to produce an α-glucan.

In one embodiment, said β-1,4-glucan may be cellobiose, and said β-1,4-glucan phosphorylase may be cellobiose phosphorylase.

In one embodiment, said β-1,4-glucan may be a cellooligosaccharide having a degree of polymerization of 3 or more, and said β-1,4-glucan phosphorylase may be cellodextrin phosphorylase.

In one embodiment, said β-1,4-glucan may be a cellooligosaccharide having a degree of polymerization of 3 or more, and said β-1,4-glucan phosphorylase may be cellobiose phosphorylase and cellodextrin phosphorylase.

In one embodiment, said production step may further include removal of glucose produced as a byproduct from the solution simultaneously with production of said α-glucan.

In one embodiment, said solution may further contain glucose isomerase or glucose oxidase.

In one embodiment, said solution may further contain glucose oxidase and mutarotase.

In one embodiment, said solution may further contain catalase or peroxidase.

In one embodiment, said source of phosphoric acid may be inorganic phosphoric acid, glucose-1-phosphate, or a mixture of inorganic phosphoric acid and glucose-1-phosphate.

In one embodiment, the concentration of said source of phosphoric acid may be 1 mM to 50 mM.

In one embodiment, the method of the invention is the method according to claim 1, wherein said α-glucan may be an amylose.

### EFFECT OF THE INVENTION

By the method of the present invention, non-digestible cellulose can be efficiently converted into a digestible food.

### BRIEF DESCRIPTION OF THE DRAWINGS

FIG. 1 shows a schematic of the reaction generated in the production method of the present invention.

FIG. 2 shows a schematic of the reaction generated in the production method of the present invention in the case where cellobiose is used as the β-1,4-glucan, and cellobiose phosphorylase is used as the β-1,4-glucan phosphorylase.

FIG. 3 shows a change in the amylose yield in the case where the concentration of cellobiose phosphorylase is changed.

FIG. 4 shows a change in the amylose yield in the case where the concentration of phosphoric acid is changed.

FIG. 5 shows a change in the amylose yield in the case where the concentration of cellobiose is increased while the ratio of the concentration of cellobiose, the concentration of primer, and the concentration of phosphoric acid is kept constant.

FIG. 6 shows a change in the amylose yield in the case where glucose isomerase (GI) or glucose oxidase (GOx)+mutarotase (MT)+peroxidase (POx) are added in the production method of the present invention.

### SEQUENCE LISTING FREE TEXT

SEQ ID NO: 1 is the base sequence of a synthetic DNA primer 1; and

SEQ ID NO: 2 is the base sequence of a synthetic DNA primer 2.

### BEST MODE FOR CARRYING OUT THE INVENTION

The present invention will be explained in detail below.

It should be understood that, unless otherwise specially referred to, an expression in a singular form also includes the concept in plural forms thereof throughout the present specification. In addition, it should be understood that, unless otherwise specially referred to, terms used in the present specification are used in the sense which is normally used in the art.

The term “α-glucan”, as used herein, refers to a saccharide containing D-glucose as a constituent unit, and having at least two or more saccharide units linked with an α-1,4-glucoside bond. An α-glucan may be a linear, branched or cyclic molecule. A linear α-glucan and α-1,4-glucan are synonymous. In a linear α-glucan, the saccharide units are linked only with an α-1,4-glucoside bond. An α-glucan containing one or more α-1,6-glucoside bonds is a branched α-glucan. An α-glucan preferably contains a linear section to some extent. A linear α-glucan having no branching is more preferable. An α-glucan produced in the present invention is preferably an amylose, a glucan having a cyclic structure or a glucan having a branched structure, more preferably an amylose. The number of saccharide units contained in one α-glucan molecule is referred to as the degree of polymerization of this α-glucan.

In some cases, it is preferable that an α-glucan has a small number of branches (i.e. number of α-1,6-glucoside bonds). In such a case, the number of branches is typically 0 to 10000, preferably 0 to 1000, more preferably 0 to 500, further preferably 0 to 100, further preferably 0 to 50, further preferably 0 to 25, further preferably 0.

In a branched α-glucan produced by the method of the present invention, the ratio of the number of α-1,4-glucoside bonds relative to the number of α-1,6-glucoside bonds, setting the number of α-1,6-glucoside bonds to be 1, is preferably 1 to 10000, more preferably 10 to 5000, further preferably 50 to 1000, further preferably 100 to 500.

α-1,6-glucosidic bonds may be distributed in an α-glucan randomly, or may be distributed uniformly. A distribution to such an extent that a linear part of 5 or more saccharide units is formed in an α-glucan is preferable.

An α-glucan may be constructed only of D-glucose, or may be a derivative modified to such an extent that the nature of such an α-glucan is not deteriorated. It is preferable that α-glucan is not modified. Modifications to such an extent that the nature of the α-glucan is not deteriorated include, but are not limited to, esterification, etherization, crosslinking and the like. These modifications may be performed according to the method known in the art.

An α-glucan has a molecular weight of typically about 1×10³ or more, preferably about 5×10³ or more, more preferably about 1×10⁴ or more, further preferably about 5×10⁴ or more, further preferably about 1×10⁵ or more. An α-glucan has a molecular weight of typically about 1×10⁶ or less, preferably about 5×10⁵ or less, further preferably about 1×10⁵ or less.

Those skilled in the art easily understand that an α-glucan having a desired molecular weight is obtained by appropriately selecting the amount of a substrate (e.g. a primer, a β-1,4-glucan etc.), the amount of an enzyme, a reaction time and the like used in the production method of the present invention.

<Materials Used in Production of α-glucan>

In the production method of the present invention, for example, a solution containing a β-1,4-glucan, a primer, a source of phosphoric acid, β-1,4-glucan phosphorylase, and α-1,4-glucan phosphorylase is used. In preparing this solution, for example, a β-1,4-glucan, a primer, inorganic phosphoric acid or glucose-1-phosphate, β-1,4-glucan phosphorylase, α-1,4-glucan phosphorylase, a buffer and solvents in which they are dissolved are used as the main materials. These materials are usually all added at reaction initiation, but any material among them may be additionally added during the reaction.

The term “source of phosphoric acid”, as used herein, refers to a molecule which can provide phosphoric acid to a catalytic reaction of CBP. Examples of a source of phosphoric acid include, but are not limited to, inorganic phosphoric acid (inorganic phosphate such as NaH₂PO₄, Na₂HPO₄, KH₂PO₄ and K₂HPO₄) and organic phosphates (e.g. glucose-1-phosphate).

In the production method of the present invention, the solution may further contain glucose isomerase or glucose oxidase. When glucose oxidase is used, mutarotase may be further contained. Further, when glucose oxidase is used, the solution in the present invention may further contain catalase or peroxidase.

In the production method of the present invention, as necessary, an enzyme selected from the group consisting of debranching enzyme, branching enzyme, 4-α-glucanotransferase and glycogen debranching enzyme may be used. An enzyme selected from the group consisting of debranching enzyme, branching enzyme, 4-α-glucanotransferase and glycogen debranching enzyme may be added to the solution from the beginning of the production method of the present invention, or maybe added to the solution during the method, depending on the structure of the objective α-glucan.

(1. β-1,4-glucan)

The term “β-1,4-glucan”, as used herein, refers to a saccharide containing D-glucose as the constituent unit, and having at least two or more saccharide units linked with an β-1,4-glucoside bond. A β-1,4-glucan may be a linear molecule. A linear β-glucan, β-1,4-glucan and cellulose are synonymous. In a linear β-glucan, saccharide units are linked only with a β-1,4-glycoside bond. The number of saccharide units contained in one β-1,4-glucan molecule refers to the degree of polymerization of this β-1,4-glucan. The degree of polymerization of β-1,4-glucan is preferably about 2 to about 10, more preferably about 2 to about 8, more preferably about 2 to about 5. β-1,4-glucan having a degree of polymerization of about 2 to about 10 is also referred to as a cellooligosaccharide. β-1,4-glucan having a degree of polymerization of 2 is particularly referred to as cellobiose. β-1,4-glucan having a degree of polymerization of 3 is referred to as cellotriose. β-1,4-glucan having a degree of polymerization of 4 is referred to as cellotetraose. Since as the degree of polymerization of β-1,4-glucan is lowered, solubility is higher, and handling is easier, β-1,4-glucan having a lower degree of polymerization is more preferable. β-1,4-glucan is present in all plants. β-1,4-glucan may be unmodified after isolation from a plant, or may be β-1,4-glucan obtained by chemically or enzymatically treating one isolated from a plant. Alternatively, β-1,4-glucan maybe cellulose reproduced from waste such as used paper, building materials, and used cloth, or may be β-1,4-glucan prepared therefrom. For example, by acting cellulase on high-molecular-weight cellulose isolated from a plant, a cellooligosaccharide having a lower molecular weight is obtained. A method for producing a large amount of cellooligosaccharides from plants is known in the art. An example of such a document includes Japanese Patent Laid-Open Publication No. 2001-95594. β-1,4-glucan may be provided at any production stage, from a crushed plant solution containing β-1,4-glucan, to purified β-1,4-glucan. It is preferable that β-1,4-glucan used in the method of the present invention is pure. However, β-1,4-glucan may contain any other contaminant as long as the action of an enzyme used in the present invention is not inhibited.

The concentration of β-1,4-glucan contained in the solution is typically about 0.1% to about 40%, preferably about 0.5% to about 30%, more preferably about 1% to about 20%, particularly preferably about 2% to about 15%, most preferably about 3% to about 12%. It should be noted that, in the present specification, the concentration of the β-1,4-glucan is calculated in Weight/Volume, that is, (weight of the β-1,4-glucan)×100/(volume of the solution). When the weight of the β-1,4-glucan is too large, unreacted β-1,4-glucan is precipitated from a solution in some cases. When the amount of a β-1,4-glucan to be used is too small, the reaction itself occurs at a high temperature, but the yield is reduced in some cases.

In the present specification, the ratio obtained by dividing the molar concentration of the β-1,4-glucan in the solution by the sum of the molar concentration of inorganic phosphoric acids and the molar concentration of glucose-1-phosphates in the reaction solution is referred to as the β-1,4-glucan:phosphoric acid ratio. That is, the ratio is as follows:
β-1,4-glucan:phosphoric acid ratio=(molar concentration of β-1,4-glucan)/(sum of molar concentration of inorganic phosphoric acids and molar concentration of glucose-1-phosphates) (Equation 1)

If all reaction materials are put in the solution to initiate the reaction, and no additional materials are added during the reaction, the β-1,4-glucan:phosphoric acid ratio is maximum at reaction initiation. The β-1,4-glucan:phosphoric acid ratio at reaction initiation can be any ratio, but preferably about 0.01 or more, more preferably about 0.03 or more, further preferably about 0.06 or more, particularly preferably about 0.1 or more, most preferably about 0.1 to about 0.6.

(2. Primer)

A primer used in the method of the present invention refers to a molecule which acts as a starting substance for adding a glycoside residue for synthesis of an α-glucan. It should be noted that, in the present specification, a glycoside residue and a glucose residue can be used interchangeably. A primer may also be said to be a molecule which acts as an acceptor for a glycoside residue of G-1-P. If a primer has one or more free moieties which can be linked with a saccharide unit by an α-1,4-glycoside bond, other parts of the primer may be form a moiety other than with a saccharide. In the method of the present invention, by transfer of one glycoside residue on a primer contained at reaction initiation with an α-1,4 bond, an α-glucan having a degree of polymerization which is larger by one than that of this primer is formed. The formed α-glucan can act again as an acceptor in the same solution. Like this, in the method of the present invention, glycoside residues are successively connected to the primer with an α-1,4-glucoside bond, thereby, an α-glucan having any degree of polymerization is synthesized. A primer includes any saccharide to which a saccharide unit can be added by glucan phosphorylase.

A primer which can act as a starting substance for the reaction of the present invention is sufficient. For example, it is also possible to again extend an α-1,4-glycoside chain according to the method of the present invention using α-glucan synthesized by the method of the present invention as a primer.

A primer may be α-1,4-glucan containing only α-1,4-glycoside bond(s), or may partially contain α-1,6-glycoside bond(s). Those skilled in the art can easily select an appropriate primer depending on the desired glucan. In the case where a linear amylose is synthesized, it is preferable to use an α-1,4-glucan containing only α-1,4-glycoside bond(s) as a primer, because a linear amylose can be synthesized without using a debranching enzyme.

Examples of a primer include a maltooligosaccharide, an amylose, an amylopectin, a glycogen, a dextrin, a pullulan, coupling sugar, a starch and a derivative thereof.

Maltooligosaccharide, as used herein, refers to a substance which is produced by dehydration condensation of about 2to about 10 glucoses, and is linked by α-1,4linkage(s). A maltooligosaccharide has preferably about 3 to about 10 saccharide units, more preferably about 4 to about 10 saccharide units, further preferably about 5 to about 10 saccharide units. Examples of maltooligosaccharides include maltooligosaccharides such as maltose, maltotriose, maltotetraose, maltopentaose, maltohexaose, maltoheptaose, maltooctaose, maltononaose, and maltodecaose. In one embodiment, the maltooligosaccharide is preferably maltotriose, maltotetraose, maltopentaose, maltohexaose or maltoheptaose, more preferably maltotetraose, maltopentaose, maltohexaose or maltoheptaose, further preferably maltotetraose. A maltooligosaccharide may be a single material, or a mixture of a plurality of maltooligosaccharides. A mixture of maltooligosaccharides is preferable because of the low cost. In one embodiment, a mixture of maltooligosaccharides contains at least one of maltotriose, maltose and glucose in addition to a maltooligosaccharide having a degree of polymerization more than the degree of polymerization of maltotetraose. Herein, a “maltooligosaccharide having a degree of polymerization more than the degree of polymerization of maltotetraose” refers to a maltooligosaccharide having a degree of polymerization of 4 or more. An maltooligosaccharide may be a linear maltooligosaccharide, or may be a branched maltooligosaccharide. A maltooligosaccharide can have a cyclic part in its molecule. In the present invention, a linear maltooligosaccharide is preferable.

An amylose is a linear molecule constructed of glucose units connected with α-1,4-linkages. An amylose is contained in natural starch.

An amylopectin is a branched molecule in which a glucose unit(s) is linked with an α-1,6 bond to glucose units which are linked with an α-1,4 bond. An amylopectin is contained in natural starch. As an amylopectin, for example, waxy corn starch, which consists of 100% amylopectin, can be used. For example, an amylopectin having a degree of polymerization of about 1×10⁵ or more can be used as a raw material.

A glycogen is one kind of glucan constructed of glucose, and is a glucan having a high frequency of branching. A glycogen is widely distributed as a storage polysaccharide for animals and plants in almost all cells in the granule state. In a plant, glycogen is present, for example, in the seed of corn and the like. In a glycogen, typically, sugar chains consisting of glucoses linked with an α-1,4-bond which have an average degree of polymerization of 12 to 18 are linked by an α-1,6-bond at a ratio of around one chain every about 3 units of glucose, to a sugar chain consisting of glucoses linked with an α-1,4-bond. In addition, similarly, a sugar chain consisting of glucoses linked by an α-1,4-bond is linked by an α-1,6-bond to a branch linked by an α-1,4-bond. For this reason, glycogen forms a network structure.

The molecular weight of a glycogen is typically about 1×10⁵ to about 1×10⁸, preferably about 1×10⁶ to about 1×10⁷.

A pullulan is a glucan having a molecular weight of about 100 thousands to about 300 thousands (e.g. about 200 thousands) in which maltotrioses are α-1,6-linked regularly and in a stepwise manner. A pullulan is produced, for example, by culturing black yeast Aureobasidium pullulans using starch as a raw material. A pullulan is available, for example, from Hayashibara Shoji Inc.

Coupling sugar is a mixture containing sucrose, glucosylsucrose, and maltosylsucrose as main components. Coupling sugar is produced, for example, by acting cyclodextrin glucanotransferase produced by Bacillus megaterium or the like on a mixed solution of sucrose and starch. Coupling sugar is available, for example, from Hayashibara Shoji Inc.

A starch is a mixture of amylose and amylopectin. As a starch, any starch can be used as long as it is commonly commercially available. The ratio of the amylose and amylopectin contained in a starch is different depending on the kind of a plant producing the starch. Almost all starches possessed by glutinous rice, glutinous corn and the like are an amylopectin. On the other hand, a starch consisting only of amyloses, containing no amylopectin, can not be obtained from a common plant.

Starch is classified into natural starch, a degraded starch and modified starch.

Natural starch is classified into tuber starch and cereal starch depending on the raw material. Examples of tuber starches include potato starch, tapioca starch, sweet potato starch, kudzu starch, bracken starch and the like. Examples of cereal starches include corn starch, wheat starch, rice starch and the like. Examples of natural starches are high amylose starches (e.g. high amylose corn starch) in which the content of amyloses is increased to 50% to 70% as a result of breeding of a plant producing starch. Other examples of natural starches include waxy starches which contain no amyloseas a result of breeding of a plant producing starch.

Soluble starch refers to water-soluble starch obtained by subjecting natural starch to various treatments.

Modified starch is a starch to which a more utilizable nature has been imparted by subjecting a natural starch to treatment such as hydrolysis, esterification or gelatinization. A wide variety of modified starches having various combinations of gelatinization initiation temperature, viscosity when formed into a paste, transparency when formed into a paste, and aging stability are available. There are many kinds of modified starches. One example of such a starch is starch in which, by immersing a starch granule in an acid at a temperature not higher than the gelatinization temperature of the starch, the starch molecule is cut, but the starch granule is not destructed.

A degraded starch is an oligosaccharide or a polysaccharide which is obtained by subjecting said starch to a treatment such as enzyme treatment or hydrolysis, and resulting in a starch which has a smaller molecular weight than before treatment. Examples of degraded starches include a starch degraded with debranching enzyme, a starch degraded with phosphorylase and a partially hydrolyzed starch.

A starch degraded with debranching enzyme is obtained by acting a debranching enzyme on starch. By variously changing the acting time of the debranching enzyme, a starch degraded with debranching enzyme in which the branch part (i.e. α-1,6-glucoside bond) has been cut to an arbitrary degree is obtained. Examples of a starch degraded with debranching enzyme include a degraded product having 1 to 20 α-1,6-glucoside bonds in a saccharide unit number of 4 to 10000, a degraded product having no α-1,6-glucoside bonds in a saccharide unit number of 3 to 500, maltooligosaccharide, and amylose. In the case of a starch degraded with debranching enzyme, the distribution of the molecular weight of the resulting degraded product may be different depending on the kind of starch to be degraded. A starch degraded with debranching enzyme may be a mixture of sugar chains of various lengths.

A starch degraded with phosphorylase is obtained by acting glucan phosphorylase (also referred to as phosphorylase) on starch. Glucan phosphorylase transfers a glucose residue saccharide units one by one from the non-reducing terminal of the starch to another substrate. Since glucan phosphorylase cannot cut α-1,6-glucoside bonds, when glucan phosphorylase is acted on starch for a sufficiently long time, a degraded product in which cleavage terminates at an α-1,6-glucoside bond is obtained. In the present invention, the number of saccharide units possessed by a starch degraded with phosphorylase is preferably about 10 to about 100,000, more preferably about 50 to about 50,000, further more preferably about 100 to about 10,000. A starch degraded with phosphorylase may have a different distribution of the molecular weight of the resulting degraded product depending on the kind of starch to be degraded. A starch degraded with phosphorylase can be a mixture of sugar chains having various lengths.

Dextrin and partially hydrolyzed starch refer to a degraded product obtained by partially degrading starch by the action of an acid, an alkyl, an enzyme or the like. In the present invention, the number of saccharide units possessed by dextrin and partially hydrolyzed starch is preferably about 10 to about 100,000, more preferably about 50 to about 50,000, further more preferably about 100 to about 10,000. In the case of dextrin and partially hydrolyzed starch, the distribution of the molecular weight of the resulting degraded product may be different depending on the kind of starch to be degraded. Dextrin and partially hydrolyzed starch can be a mixture of saccharide chains having various lengths.

It is preferable that the starch is selected from the group consisting of a soluble starch, a waxy starch, a high amylose starch, a starch degraded with debranching enzyme, a starch degraded with phosphorylase, a partially hydrolyzed starch, a modified starch, and a derivative thereof.

In the method of the present invention, derivatives of the aforementioned various saccharides can be used as primers. For example, derivatives in which at least one of the alcoholic hydroxy groups of the aforementioned saccharides is hydroxyalkylated, alkylated, acetylated, carboxymethylated, sulfated, or phosphorylated can be used. Further, a mixture of two or more kinds of these derivatives can be used as a raw material.

(3. Inorganic Phosphoric Acid or glucose-1-phosphate)

In the present specification, a source of phosphoric acid such as inorganic phosphoric acid refers to a substance which can donate a phosphoric acid substrate in a reaction with CBP. Herein, a phosphoric acid substrate refers to a substance which is a raw material for the phosphoric acid moiety of glucose-1-phosphate. In β-1,4-glucan phosphorolysis catalyzed by β-1,4-glucan phosphorylase, it is thought that inorganic phosphoric acid acts as a substrate in the form of a phosphate ion. Since this substrate is conventionally referred to as inorganic phosphoric acid in the art, this substrate is referred to as inorganic phosphoric acid also in the present specification. Inorganic phosphoric acid includes phosphoric acid and an inorganic salt of phosphoric acid. Usually, inorganic phosphoric acid is used in water containing a cationic ion such as an alkali metal ion. In this case, since phosphoric acid, phosphate and the phosphate ions are brought into a state of equilibrium, it is difficult to differentiate phosphoric acid and phosphate. Therefore, conveniently, phosphoric acid and phosphate are collectively referred to as inorganic phosphoric acid. In the present invention, inorganic phosphoric acid is preferably any metal salt of phosphoric acid, more preferably an alkali metal salt of phosphoric acid. Preferable specific examples of inorganic phosphoric acid include sodium dihydrogen phosphate, disodium hydrogen phosphate, trisodium phosphate, potassium dihydrogen phosphate, dipotassium hydrogen phosphate, tripotassium phosphate, phosphoric acid (H₃PO₄), ammonium dihydrogen phosphate, diammonium hydrogen phosphate and the like.

Only one kind of, or a plurality of kinds of inorganic phosphoric acids, maybe contained in a CBP-GP reaction system at reaction initiation.

Inorganic phosphoric acid can be provided, for example, by degrading a phosphoric acid condensate such as polyphosphoric acid (e.g. pyrophosphoric acid, triphosphoric acid and tetraphosphoric acid) or a salt thereof, by a physical, chemical or enzymatic reaction, and adding this to the reaction solution.

In the present specification, glucose-1-phosphate refers to glucose-1-phosphoric acid (C₆H₁₃O₉P) and a salt thereof. Glucose-1-phosphate is preferably any metal salt of glucose-1-phosphoric acid (C₆H₁₃O₉P) in a narrow sense, more preferably any alkali metal salt of glucose-1-phosphoric acid (C₆H₁₃O₉P). Preferable specific examples of glucose-1-phosphate include disodium glucose-1-phosphate, dipotassium glucose-1-phosphate, glucose-1-phosphoric acid (C₆H₁₃O₉P) and the like. In the present specification, a glucose-1-phosphate whose chemical formula is not drawn in parentheses indicates glucose-1-phosphate in a wide sense, that is, glucose-1-phosphoric acid (C₆H₁₃O₉P) in a narrow sense and a salt thereof.

Only one kind of, or a plurality of kinds of glucose-1-phosphates, maybe contained in the CBP-GP reaction system at reaction initiation.

In the method of the present invention, the ratio of phosphoric acid and glucose-1-phosphate in the reaction solution at reaction initiation may be any ratio.

The sum of the molar concentration of inorganic phosphoric acid and the molar concentration of glucose-1-phosphate contained in a reaction solution is typically about 0.1 mM to about 1000 mM, preferably about 1 mM to about 500 mM, more preferably about 1 mM to about 50 mM, further more preferably about 5 mM to about 30 mM. When the amounts of inorganic phosphoric acid and glucose-1-phosphate are too large, the reaction itself occurs, but the yield of the α-glucan is reduced in some cases. When the amounts of them to be used are too small, it takes a long time for synthesizing α-glucan in some cases.

The content of inorganic phosphoric acid in a solution in the method of the present invention can be quantitated by the methods known in the art. The content of glucose-1-phosphate in this solution can be quantitated by the methods known in the art. When a phosphorus-containing substance not involved in the reaction is not used, in such case, the total content of inorganic phosphoric acid and glucose-1-phosphate may be measured by an atomic absorption method.

Inorganic phosphoric acid is obtained, for example, as phosphate ions, by the following method. Into a solution (200 μl) containing inorganic phosphoric acid is mixed 800 μl of a molybdenum reagent (15 mM ammonium molybdate, 100 mM zinc acetate), subsequently, 200 μl of 568 mM ascorbic acid (pH 5.0) is added, and this is stirred to obtain a reaction system. After this reaction system is retained at 30° C. for 20 minutes, an absorbance at 850 nm is measured using a spectrophotometer. Absorbance is similarly measured using an inorganic phosphoric acid of known concentration to produce a standard curve. The absorbance obtained for the sample is fitted to this standard curve to obtain inorganic phosphoric acid in a sample. In this quantitation method, the amount of inorganic phosphoric acid is quantitated, and the amount of glucose-1-phosphate is not quantitated.

Glucose-1-phosphate can be quantitated, for example, by the following method. To 300 μl of measuring reagent (200 mM Tris-HCl (pH 7.0), 3 mM NADP, 15 mM magnesium chloride, 3 mM EDTA, 15 μM glucose-1,6-diphosphoric acid, 6 μg/ml phosphoglucomutase, 6 μg/ml glucose-6-phosphate dehydrogenase), 600 μl of a solution containing appropriately diluted glucose-1-phosphate is added, and this is stirred to obtain a reaction system. After this reaction system is retained at 30° C. for 30 minutes, an absorbance at 340 nm is measured using a spectrophotometer. Absorbance is similarly measured using sodium glucose-1-phosphate of known concentration, and a standard curve is produced. The absorbance obtained for the sample is fitted to this standard curve to obtain the glucose-1-phosphate concentration in the sample. Usually, the activity of producing one μmol glucose-1-phosphate for 1 minute is defined as one unit. In this quantitation method, only glucose-1-phosphate is quantitated, and the amount of inorganic phosphoric acid is not quantitated.

(4. β-1,4-Glucan Phosphorylase)

The “β-1,4-glucan phosphorylase”, as used herein, refers to any enzyme which performs phosphorolysis by transferring a glucose residue at the non-reducing terminal of a β-1,4-glucan to a phosphate group. β-1,4-glucan phosphorylase can also catalyze a reaction synthesizing a β-1,4-glucan, which is the reverse reaction of phosphorolysis. Whether the reaction proceeds in either direction depends on amounts of substrates, but this reaction tends to easily proceed in the direction of the reaction synthesizing a β-1,4-glucan. The reaction catalyzed by β-1,4-glucan phosphorylase is shown by the following formula:
β-1,4-glucan (degree of polymerization n)+inorganic phosphoric acid [symbol] β-1,4-glucan (degree of polymerization n−1)+α-D-glucose-1-phosphate (Chemical formula 1)

It should be noted that, in this formula, when the degree of polymerization of the β-1,4-glucan at the beginning is 2, glucose is obtained in place of β-1,4-glucan.

β-1,4-glucan phosphorylase is preferably cellobiose phosphorylase (EC: 2.4.1.20) or cellodextrin phosphorylase (EC: 2.4.1.49).

Cellobiose phosphorylase refers to an enzyme which performs phosphorolysis by transferring a glucose residue at the non-reducing terminal of cellobiose to a phosphate group. The reaction catalyzed by cellobiose phosphorylase is shown by the following formula:
cellobiose+inorganic phosphoric acid [symbol] glucose+α-D-glucose-1-phosphate (Chemical formula 2)

Cellodextrin phosphorylase refers to an enzyme which performs phosphorolysis by transferring a glucose residue at the non-reducing terminal of a cellooligosaccharide having a degree of polymerization of 3 or more to a phosphate group. Cellooligosaccharide is also referred to as cellodextrin. The reaction catalyzed by cellodextrin phosphorylase is shown by the following formula:
cellooligosaccharide (degree of polymerization n)+inorganic phosphoric acid [symbol] cellooligosaccharide (degree of polymerization n−1)+α-D-glucose-1-phosphate (Chemical formula 3)

In the method of the present invention, when the β-1,4-glucan is cellobiose, it is preferable to use cellobiose phosphorylase as the β-1,4-glucan phosphorylase. In the method of the present invention, when the β-1,4-glucan is cellooligosaccharide, it is preferable to use cellodextrin phosphorylase as the β-1,4-glucan phosphorylase. In addition, in the method of the present invention, when the β-1,4-glucan is cellooligosaccharide, it is preferable to use cellobiose phosphorylase and cellodextrin phosphorylase as the β-1,4-glucan phosphorylase. In this case, since glucose-1-phosphate produced by degrading cellooligosaccharide by the action of cellodextrin phosphorylase is used in synthesizing the α-glucan, and finally a cellobiose is produced that can be degraded with cellobiose phosphorylase, the rate of synthesizing an α-glucan from a cellooligosaccharide becomes higher.

β-1,4-glucan phosphorylase is contained in various organisms in the natural world. Examples of an organism producing β-1,4-glucan phosphorylase include an organism of the genus Clostridium (e.g. Clostridium thermocellum and Clostridium sterocorarium), an organism of the genus Cellvibrio (e.g. Cellvibrio gilvus), an organism of the genus Thermotoga (e.g. Thermotoga neapolitana and Thermotoga maritima), an organism of the genus Ruminococcas (e.g. Ruminococcas flavofaciens), an organism of the genus Fomes (e.g. Fomes annos), an organism of the genus Cellulomonas and an organism of the genus Erwinia. An organism producing β-1,4-glucan phosphorylase is preferably selected from the group consisting of Clostridium thermocellum, Clostridium sterocorarium, Cellvibrio gilvus, Thermotoga neapolitana, Thermotoga maritima, Ruminococcas flavofaciens, Fomes annos, Cellulomonas sp., and Erwinia sp. β-1,4-glucan phosphorylase may be derived from a plant.

Cellobiose phosphorylase is contained in various organisms in the natural world. Examples of an organism producing cellobiose phosphorylase include an organism of the genus Clostridium (e.g. Clostridium thermocellum and Clostridium sterocorarium), an organism of the genus Cellvibrio (e.g. Cellvibrio gilvis), an organism of the genus Thermotoga (e.g. Thermotoga neapolitana and Thermotoga maritima), an organism of the genus Ruminococcas (e.g. Ruminococcas flavofaciens), an organism of the genus Fomes (e.g. Fomes annos), an organism of the genus Cellulomonas and an organism of the genus Erwinia. An organism producing cellobiose phosphorylase is preferably selected from the group consisting of Clostridium thermocellum, Clostridium sterocorarium, Cellvibrio gilvis, Thermotoga neapolitana, Thermotoga maritima, Ruminococcas flavofaciens, Fomes annos, Cellulomonas sp., and Erwinia sp., more preferably Clostridium thermocellum or Cellvibrio gilvus, most preferably Clostridium thermocellum. Cellobiose phosphorylase may be derived from a plant.

Cellodextrin phosphorylase is contained in various organisms in natural world. Examples of an organism producing cellodextrin phosphorylase include an organism of the genus Clostridium (e.g. Clostridium thermocellum and Clostridium sterocorarium), an organism of the genus Cellvibrio (e.g. Cellvibrio gilvus), an organism of the genus Thermotoga (e.g. Thermotoga neapolitana and Thermotoga maritima), an organism of the genus Ruminococcas (e.g. Ruminococcas flavofaciens), an organism of the genus Fomes (e.g. Fomes annos), an organism of the genus Cellulomonas and an organism of the genus Erwinia. An organism producing cellodextrin phosphorylase is preferably selected from the group consisting of Clostridium thermocellum, Clostridium sterocorarium, Cellvibrio gilvus, Thermotoga neapolitana, Thermotoga maritima, Ruminococcas flavofaciens, Fomes annos, Cellulomonas sp., and Erwinia sp., more preferably Clostridium thermocellum or Cellulomonas sp., most preferably Clostridium thermocellum. Cellodextrin phosphorylase may be derived from a plant.

β-1,4-glucan phosphorylase (preferably cellobiose phosphorylase or cellodextrin phosphorylase, most preferably cellobiose phosphorylase) can be derived from any organism producing β-1,4-glucan phosphorylase (preferably cellobiose phosphorylase or cellodextrin phosphorylase, most preferably cellobiose phosphorylase). It is preferable that the β-1,4-glucan phosphorylase has thermostability to some extent. It is more preferable that β-1,4-glucan phosphorylase has high thermostability. For example, when β-1,4-glucan phosphorylase is heated at 55° C. for 20 minutes in a 50 mM phosphate buffer (pH 7.5) containing 1.4 mM 2-mercaptoethanol, preferably 50% or more, more preferably 60% or more, further preferably 70% or more, particularly preferably 80% or more, most preferably 85% or more of the activity of β-1,4-glucan phosphorylase before heating is retained. β-1,4-glucan phosphorylase is preferably derived from a bacterium selected from the group consisting of Clostridium thermocellum, Clostridium sterocorarium, Cellvibrio gilvus, Thermotoga neapolitana, Thermotoga maritima, Ruminococcas flavofaciens, Fomes annos, Cellulomonas sp., and Erwinia sp.

When β-1,4-glucan phosphorylase is cellobiose phosphorylase, cellobiose phosphorylase is preferably derived from a bacterium selected from the group consisting of Clostridium thermocellum, Clostridium sterocorarium, Cellvibrio gilvus, Thermotoga neapolitana, Thermotoga maritima, Ruminococcas flavofaciens, Fomes annos, Cellulomonas sp., and Erwinia sp., more preferably is derived from Clostridium thermocellum or Cellvibrio gilvus, most preferably is derived from Clostridium thermocellum.

When the β-1,4-glucan phosphorylase is cellobiose phosphorylase, the cellobiose phosphorylase is preferably derived from a bacterium selected from the group consisting of Clostridium thermocellum, Clostridium sterocorarium, Cellvibrio gilvus, Thermotoga neapolitana, Thermotoga maritima, Ruminococcas flavofaciens, Fomes annos, Cellulomonas sp., and Erwinia sp., more preferably is derived from Clostridium thermocellum or Cellulomonas sp., most preferably is derived from Clostridium thermocellum.

In the present specification, that an enzyme is “derived” from an organism means not only that the enzyme is directly isolated from the organism, but also that the enzyme is obtained by utilizing the organism in any form. For example, when a gene encoding the enzyme obtained from the organism is introduced into Escherichia coli, and an enzyme is isolated from the Escherichia coli, the enzyme is “derived” from the organism.

β-1,4-glucan phosphorylase used in the present invention can be directly isolated from an organism producing β-1,4-glucan phosphorylase, such as the aforementioned organisms, present in the natural world. β-1,4-glucan phosphorylase used in the present invention may be isolated from a microorganism (e.g. bacteria, fungi etc.) which has been genetically modified with a gene encoding β-1,4-glucan phosphorylase isolated from the aforementioned organism.

β-1,4-glucan phosphorylase used in the method of the present invention can be prepared, for example, as follows. First, a microorganism (e.g. bacteria, fungi etc.) producing β-1,4-glucan phosphorylase is cultured. This microorganism may be a microorganism directly producing the β-1,4-glucan phosphorylase. Alternatively, a gene encoding the β-1,4-glucan phosphorylase may be cloned, a microorganism (e.g. bacteria, fungi etc.) which is advantageous for expressing the β-1,4-glucan phosphorylase may be genetically modified with the resulting gene to obtain a recombinant microorganism, and β-1,4-glucan phosphorylase may be obtained from the resulting microorganism.

A microorganism used in genetic modification with β-1,4-glucan phosphorylase gene can be easily selected, taking various conditions such as ease of expression of the β-1,4-glucan phosphorylase, ease of culturing, proliferation speed and safety into consideration. Since the β-1,4-glucan phosphorylase preferably contains no amylase as a contaminant, it is preferable to use a microorganism (e.g. bacteria, fungi etc.) which does not produce amylase or expresses amylase only at a low level, for genetic modification. For genetic modification with β-1,4-glucan phosphorylase, it is preferable to use a mesophilic microorganism such as Escherichia coli or Bacillus subtilis. β-1,4-glucan phosphorylase produced using a microorganism (e.g. bacteria, fungi etc.) which does not produce amylase or expresses amylase only at a low level containing substantially no amylase is preferably used in the method of the present invention.

Genetic modification of a microorganism (e.g. bacteria, fungi etc.) with a cloned gene can be performed according to methods well-known to those skilled in the art. When a cloned gene is used, it is preferable to operably link this gene to a constitutive promoter or an inducible promoter. The “operably linked” refers to that a promoter and a gene are linked so that expression of the gene is regulated by the promoter. When an inducible promoter is used, it is preferable to perform culturing under inducing conditions. Various inducible promoters are known to those skilled in the art.

Regarding a cloned gene, a base sequence encoding a signal peptide can be linked to this gene so that produced β-1,4-glucan phosphorylase is secreted outside the bacterium. A base sequence encoding a signal peptide is known to those skilled in the art.

Those skilled in the art can appropriately set conditions for culturing microorganisms (e.g. bacteria, fungi etc.) in order to produce the β-1,4-glucan phosphorylase. An appropriate medium for culturing the microorganism, the appropriate conditions for inducing each inducible promoter and the like are known to those skilled in the art.

For example, when expressed β-1,4-glucan phosphorylase is accumulated in a transformed cell, after the transformed cell is cultured under the appropriate conditions, the cells are recovered by centrifuging or filtering the culture, and then are suspended in an appropriate buffer. Then, the cells are destructed by sonication or the like, and are subsequently centrifuged or filtered to obtain the supernatant. Alternatively, when expressed β-1,4-glucan phosphorylase is secreted outside a transformed cell, after the transformed cell is cultured in this manner, the cells are separated by centrifuging or filtering the culture to obtain the supernatant. When β-1,4-glucan phosphorylase is accumulated in a transformed cell, and when β-1,4-glucan phosphorylase is secreted outside a transformed cell, the thus obtained β-1,4-glucan phosphorylase-containing supernatant is concentrated using conventional means (e.g. salting out method, solvent precipitation, or ultrafiltration) to obtain the fraction containing the β-1,4-glucan phosphorylase. This fraction is filtered, or subjected to treatment such as centrifugation, desalting treatment or the like to obtain the crude enzyme solution. Further, by subjecting this crude enzyme solution to an appropriate combination of conventional enzyme purifying means such as lyophilization, isoelectric focusing, ion exchange chromatography, and crystallization, the crude enzyme or purified enzyme having improved specific activity is obtained. When no enzymes hydrolyzing a glucan such as α-amylase are contained, the crude enzyme can be used as is for producing, for example, an α-glucan.

The amount of β-1,4-glucan phosphorylase contained in the solution at reaction initiation is typically about 0.01 to 1,000 U/g β-1,4-glucan, preferably about 0.05 to 500 U/g β-1,4-glucan, more preferably about 0.1 to 100 U/g β-1,4-glucan, particularly preferably about 0.5 to 50 U/g β-1,4-glucan, most preferably about 1 to 7 U/g β-1,4-glucan per β-1,4-glucan in a solution at reaction initiation. When the weight of the β-1,4-glucan phosphorylase is too large, the denatured enzyme is easily aggregated during reaction in some cases. When the amount to be used is too small, the reaction itself occurs, but the yield of the glucan is reduced in some cases.

The β-1,4-glucan phosphorylase may be purified or unpurified. The β-1,4-glucan phosphorylase may be immobilized or may not be immobilized. It is preferable that the β-1,4-glucan phosphorylase is immobilized. As the method of immobilization, methods well-known to those skilled in the art such as a carrier binding method (e.g. covalent binding method, ion binding method, or physical adsorbing method), a crosslinking method or an inclusion method (lattice type or microcapsule type) can be used. It is preferable that the β-1,4-glucan phosphorylase is immobilized on a carrier.

(5. α-1,4-Glucan Phosphorylase)

α-1,4-glucan phosphorylase (EC: 2.4.1.1) is a generic name of enzymes which catalyze production of α-1,4-glucan (degree of polymerization n−1) and α-D-glucose-1-phosphate by phosphorolysis of α-1,4-glucan (degree of polymerization n), and is also called phosphorylase, starch phosphorylase, glycogen phosphorylase, maltodextrin phosphorylase and the like in some cases. Glucan phosphorylase can also catalyze a reaction synthesizing α-1,4-glucan (degree of polymerization n) from α-1,4-glucan (degree of polymerization n−1) and α-D-glucose-1-phosphate, which is the reverse reaction of phosphorolysis. Whether a reaction proceeds in either direction depends on the amounts of substrates. In vivo, since the amounts of inorganic phosphoric acids are large, glucan phosphorylase progresses a reaction to the direction of phosphorolysis. In the method of the present invention, since inorganic phosphoric acid is used in phosphorolysis of β-1,4-glucan, and the amounts of inorganic phosphoric acids contained in the reaction solution are small, the reaction proceeds to the direction of synthesis of α-glucan.

It is thought that α-1,4-glucan phosphorylase is universally present in various plants, animals and microorganisms which can store starch or glycogen.

Examples of plants producing α-1,4-glucan phosphorylase include algae; tubers such as potato (also referred to as Irish potato), sweet potato (also referred to as Ipomoea batatas), yam, taro, and cassava; vegetables such as cabbage and spinach, cereals such as corn, rice, wheat, barley, rye, and foxtail millet; beans such as pea, soybean, adzuki bean, and mottled kidney bean; and the like.

Examples of an animal producing α-1,4-glucan phosphorylase include mammals such as human, rabbit, rat and pig.

Examples of microorganisms producing α-1,4-glucan phosphorylase include Thermus aquaticus, Bacillus stearothermophilus, Deinococcus radiodurans, Thermococcus litoralis, Streptomyces coelicolor, Pyrococcus horikoshi, Mycobacterium tuberculosis, Thermotoga maritima, Aquifex aeolicus, Methanococcus Jannaschii, Pseudomonas aeruginosa, Chlamydia pneumoniae, Chlorella vulgaris, Agrobacterium tumefaciens, Clostridium pasteurianum, Klebsiella pneumoniae, Synecococcus sp., Synechocystis sp., E. coli, Neurospora crassa, Saccharomyces cerevisiae, and Chlamydomonas sp. A microorganism producing α-1,4-glucan phosphorylase is not limited to these.

The α-1,4-glucan phosphorylase used in the present invention is preferably derived from potato, Thermus aquaticus, or Bacillus stearothermophilus, more preferably potato. It is preferable that α-1,4-glucan phosphorylase used in the present invention has a high optimal reaction temperature. α-1,4-glucan phosphorylase having a high optimal reaction temperature can be derived, for example, from an extreme thermophilic bacterium.

The α-1,4-glucan phosphorylase used in the present invention can be directly isolated from the aforementioned animals, plants and microorganisms which produce α-1,4-glucan phosphorylase, which are present in the natural world.

The α-1,4-glucan phosphorylase used in the present invention may be isolated from a microorganism (e.g. bacteria, fungi etc.) which has been genetically modified using a gene encoding α-1,4-glucan phosphorylase isolated from these animals, plants or microorganisms.

The α-1,4-glucan phosphorylase can be obtained from a genetically modified microorganism as in the aforementioned β-1,4-glucan phosphorylase.

A microorganism (e.g. bacteria, fungi etc.) used in genetic modification can be easily selected, taking various conditions into consideration such as ease of expression of the α-1,4-glucan phosphorylase, ease of culturing, proliferation speed, and safety, as in the aforementioned β-1,4-glucan phosphorylase. Since it is preferable that the α-1,4-glucan phosphorylase contain no amylase as a contaminant, it is preferable to use a microorganism (e.g. bacteria, fungi etc.) which does not produce amylase or expresses amylase at a low level, in genetic modification. For genetic modification with α-1,4-glucan phosphorylase, it is preferable to use a mesophilic microorganism such as Escherichia coli or Bacillus subtilis. α-1,4-glucan phosphorylase produced using a microorganism (e.g. bacteria, fungi etc.) which does not produce amylase or expresses amylase at a low level containing substantially no amylase is preferably used in the method of the present invention.

Production and purification of the α-1,4-glucan phosphorylase obtained by genetic modification can be performed as in the aforementioned β-1,4-glucan phosphorylase.

The amount of α-1,4-glucan phosphorylase contained in the solution at reaction initiation is typically about 0.05 to 1,000 U/g β-1,4-glucan, preferably about 0.1 to 500 U/g β-1,4-glucan, more preferably about 0.5 to 100 U/g β-1,4-glucan, particularly preferably about 1 to 80 U/g β-1,4-glucan, most preferably about 10 to 50 U/g β-1,4-glucan, per β-1,4-glucan in a solution at reaction initiation. When the weight of α-1,4-glucan phosphorylase is too large, enzyme denatured during the reaction is easily aggregated in some cases. When the amount to be used is too small, the reaction itself occurs, but the yield of the glucan is reduced in some cases.

The α-1,4-glucan phosphorylase may be purified, or unpurified. The α-1,4-glucan phosphorylase may be immobilized, or may not be immobilized. It is preferable that the α-1,4-glucan phosphorylase is immobilized. As the method of immobilization, methods well-known to those skilled in the art such as a carrier binding method (e.g. covalent binding method, ion binding method, or physical adsorption method), a crosslinking method or an inclusion method (lattice type or microcapsule type) can be used. It is preferable that the α-1,4-glucan phosphorylase is immobilized on a carrier. Further, the α-1,4-glucan phosphorylase may be immobilized on the same carrier as that of the β-1,4-glucan phosphorylase, or may be immobilized on another carrier. It is preferable that it is immobilized on the same carrier.

(6. Glucose isomerase (EC: 5.3.1.5))

In the production method of the present invention, it is preferable that glucose isomerase is further contained in the solution. By containing glucose isomerase in the solution, glucose produced by phosphorolysis of cellobiose can be converted into fructose. Since glucose inhibits the reaction in the direction of phosphorolysis of cellobiose, by containing glucose isomerase in the solution, phosphorolysis of cellobiose can be further promoted, and the yield of α-glucan finally obtained can be improved.

Glucose isomerase which can be used in the production method of the present invention is an enzyme which can catalyze interconversion between D-glucose and D-fructose. Since glucose isomerase can also catalyze interconversion between D-xylose and D-xylulose, it is also called xylose isomerase.

Glucose isomerase is present in microorganisms, animals and plants. Examples of microorganisms producing glucose isomerase include Streptomyces rubiginosus, Streptomyces olivochromogenes, Streptomyces murinus, Streptomyces vionaceoniger, Streptomyces diastaticus, Streptomyces albus, Streptomyces sp., Escherichia coli, Bacteroides xylanolyticus, Arthrobacter sp., Candida boidinii, Clostridium thermosulfurogenes, Clostridium thermohydrosulfuricum, Thermoanaerobacterium saccharolyticum, Thermoanaerobacter sp., Thermotoga neapolitana, Thermus aquaticus, Lactobacillus brevis, Lactobacillus xylosus, Agrobacterium tumefaciens, Bacillus sp., Actinoplanes missouriensis and Paracolobacterium aerogenoides. Examples of animals producing glucose isomerase include Trypanosoma brucei. Glucose isomerase may be derived from a plant. An organism producing glucose isomerase is not limited to these.

The glucose isomerase which can be used in the present invention is preferably derived from Streptomyces rubiginosus or Bacillus sp., more preferably Streptomyces rubiginosus. It is preferable that the glucose isomerase used in the present invention has a high optimal reaction temperature. Glucose isomerase having a high optimal reaction temperature can be derived, for example, from an extreme thermophilic bacterium.

The glucose isomerase which can be used in the present invention can be directly isolated from the aforementioned organism producing glucose isomerase, which is present in the natural world.

The glucose isomerase which can be used in the present invention may be isolated from a microorganism (e.g. bacteria, fungi etc.) which has been genetically modified using a gene encoding glucose isomerase isolated from these organisms.

The glucose isomerase can be obtained from a genetically modified microorganism as in the aforementioned β-1,4-glucan phosphorylase.

A microorganism (e.g. bacteria, fungi etc.) used in genetic modification can be easily selected, taking various conditions into consideration, such as ease of expression of glucose isomerase, ease of culturing, proliferation speed, and safety, as in the aforementioned β-1,4-glucan phosphorylase. Since it is preferable that glucose isomerase contain no amylase as a contaminant, it is preferable to use a microorganism (e.g. bacteria, fungi etc.) which does not produce amylase or expresses amylase only at a low level, for genetic modification. For genetic modification of glucose isomerase, it is preferable to use a mesophilic microorganism such as Escherichia coli or Bacillus subtilis. Glucose isomerase produced using a microorganism (e.g. bacteria, fungi etc.) which does not produce amylase or expresses amylase only at a low level containing substantially no amylase is preferably used in the method of the present invention.

Production and purification of glucose isomerase by genetic modification can be performed as in the aforementioned β-1,4-glucan phosphorylase.

The amount of glucose isomerase contained in the solution at reaction initiation is typically about 0.01 to 500 U/g β-1,4-glucan, preferably about 0.05 to 100 U/g β-1,4-glucan, more preferably about 0.1 to 50 U/g β-1,4-glucan, particularly preferably about 0.5 to 10 U/g β-1,4-gucan, most preferably about 1 to 5 U/g β-1,4-glucan, per β-1,4-glucan in the solution at reaction initiation. When the weight of glucose isomerase is too large, enzyme denatured during the reaction is easily aggregated in some cases. When the amount to be used is too small, the reaction itself occurs, but the yield of glucan is reduced in some cases.

The glucose isomerase may be purified, or may be unpurified. The glucose isomerase may be immobilized, or may not be immobilized. It is preferable that the glucose isomerase is immobilized. As the method of immobilization, methods well-known to those skilled in the art such as a carrier binding method (e.g. covalent binding method, ion binding method, or physical adsorbing method), a crosslinking method or an inclusion method (lattice type or microcapsule type) can be used. It is preferable that the glucose isomerase is immobilized on a carrier. Further, the glucose isomerase may be immobilized on the same carrier as that for at least one of the β-1,4-glucan phosphorylase and the α-1,4-glucan phosphorylase, or may be immobilized on another carrier. It is preferable that it is immobilized on the same carrier as that for both of β-1,4-glucan phosphorylase and α-1,4-glucan phosphorylase.

(7. Glucose Oxidase)

In the production method of the present invention, it is preferable that glucose oxidase is further contained in the solution. By containing glucose oxidase in the reaction solution, β-glucose which has been naturally converted from α-glucose produced by phosphorolysis of cellobiose can be converted into β-glucono-δ lactone. Since α-glucose inhibits the reaction to the direction of phosphorolysis of cellobiose, by containing glucose oxidase in the solution, phosphorolysis of cellobiose can be further promoted, and the yield of α-glucan finally obtained can be improved.

Glucose oxidase which can be used in the production method of the present invention is an enzyme which can catalyze the following reaction:
β-D-glucose+H₂O+FAD+½O₂→D-glucono-δ lactone+H₂O₂+FADH₂ (Chemical formula 4)

Glucose oxidase is present in microorganisms and plants. Examples of microorganisms producing glucose oxidase include Aspergillus niger, Penicillium amagasakiense, Penicillium notatum and Phanerochaete chrysosporium. Glucose oxidase maybe derived from a plant. An organism producing glucose oxidase is not limited to these.

The glucose oxidase which can be used in the present invention is preferably derived from Aspergillus niger or Penicillium amagasakiense, more preferably Aspergillus niger. It is preferable that the glucose oxidase used in the present invention have a high optimal reaction temperature. Glucose oxidase having a high optimal reaction temperature can be derived, for example, from an extreme thermophilic bacterium.

The glucose oxidase which can be used in the present invention can be directly isolated from the aforementioned organisms producing glucose oxidase, which are present in the natural world.

The glucose oxidase which can be used in the present invention may be isolated from a microorganism (e.g. bacteria, fungi etc.) which has been genetically modified using a gene encoding glucose oxidase isolated from these organisms.

The glucose oxidase can be obtained from a genetically modified microorganism, as in the aforementioned β-1,4-glucan phosphorylase.

A microorganism (e.g. bacteria, fungi etc.) used in genetic modification can be easily selected, taking various conditions into consideration such as ease of expression of glucose oxidase, ease of culturing, proliferation speed, and safety, as in the aforementioned β-1,4-glucan phosphorylase. Since it is preferable that glucose oxidase contain no amylase as a contaminant, it is preferable to use a microorganism (e.g. bacteria, fungi etc.) which does not produce amylase or expresses amylase only at a low level, for genetic modification. For genetic modification of glucose oxidase, it is preferable to use a mesophilic microorganism such as Escherichia coli or Bacillus subtilis. Glucose oxidase produced using a microorganism (e.g. bacteria, fungi etc.) which does not produce amylase or expresses amylase only at a low level containing substantially no amylase is preferably used in the method of the present invention.

Production and purification of glucose oxidase by genetic modification can be performed, as in the aforementioned β-1,4-glucan phosphorylase.

The amount of glucose oxidase contained in the solution at reaction initiation is typically about 0.5 to 1,000 U/g β-1,4-glucan, preferably about 1 to 500 U/g β-1,4-glucan, more preferably about 5 to 400 U/g β-1,4-glucan, particularly preferably about 10 to 300 U/g β-1,4-glucan, most preferably about 20 to 200 U/g β-1,4-glucan, per β-1,4-glucan in the solution at reaction initiation. When the weight of glucose oxidase is too large, enzyme denatured during the reaction is easily aggregated in some cases. When the amount to be used is too small, the reaction itself occurs, but the yield of glucan is reduced in some cases.

The glucose oxidase may be purified, or may be unpurified. The glucose oxidase may be immobilized, or may be not immobilized. It is preferable that the glucose oxidase is immobilized. As the method of immobilization, methods well-known to those skilled in the art such as a carrier binding method (e.g. covalent binding method, ion binding method, or physical adsorbing method), a crosslinking method or an inclusion method (lattice type or microcapsule type) can be used. It is preferable that the glucose oxidase is immobilized on a carrier. Further, the glucose oxidase may be immobilized on the same carrier as that for at least one of β-1,4-glucan phosphorylase and α-1,4-glucan phosphorylase, or may be immobilized on another carrier. It is preferable that the glucose oxidase is immobilized on the same carrier as that for both of β-1,4-glucan phosphorylase and α-1,4-glucan phosphorylase.

(8. Mutarotase)

In the production method of the present invention, when glucose oxidase is contained in the solution, it is preferable that mutarotase is further contained in the solution. By containing mutarotase in the solution, α-glucose and β-glucose produced by phosphorolysis of cellobiose can be converted into each other. Although α-glucose and β-glucose are naturally converted into each other without adding mutarotase, since interconversion is promoted by adding mutarotase, the efficient decrease in the amount of α-glucose produced by the reaction from the solution can be further improved. For that reason, by containing glucose oxidase and mutarotase in the reaction solution, the α-glucose concentration in the reaction solution is reduced, as a result, phosphorolysis of cellobiose can be further promoted, and the yield of α-glucan finally obtained can be improved.

The mutarotase which can be used in the production method of the present invention is an enzyme which can catalyze interconversion of α-glucose and β-glucose.

Mutarotase is present in microorganisms, animals and plants. Examples of microorganisms producing mutarotase include Penicillium notatum and Escherichia coli. Examples of animals producing mutarotase include pig and Bos taurus. Examples of plants producing mutarotase include Capsicum frutescens. An organism producing mutarotase is not limited to these.

The mutarotase which can be used in the present invention is preferably derived from pig or Bos taurus, more preferably pig. It is preferable that mutarotase used in the present invention has a high optimal reaction temperature. Mutarotase having a high optimal reaction temperature can be derived, for example, from an extreme thermophilic bacterium.

The mutarotase which can be used in the present invention can be directly isolated from the aforementioned organisms producing mutarotase, which are present in the natural world.

The mutarotase which can be used in the present invention may be isolated from a microorganism (e.g. bacteria, fungi etc.) which has been genetically modified using a gene encoding mutarotase isolated from these organisms.

The mutarotase can be obtained from a genetically modified microorganism, as in the aforementioned β-1,4-glucan phosphorylase.

A microorganism (e.g. bacteria, fungi etc.) used in genetic modification can be easily selected, taking various conditions into consideration such as ease of expression of mutarotase, ease of culturing, proliferation speed, and safety, as in the aforementioned β-1,4-glucan phosphorylase. Since it is preferable that the mutarotase contain no amylase as a contaminant, it is preferable to use a microorganism (e.g. bacteria, fungi etc.) which does not produce amylase or expresses amylase only at a low level for genetic modification. For genetic modification of mutarotase, it is preferable to use a mesophilic microorganism such as Escherichia coli or Baccilus subtilis. Mutarotase produced using a microorganism (e.g. bacteria, fungi etc.) which does not produce amylase or expresses amylase only at a low level containing substantially no amylase is preferably used in the method of the present invention.

Production and purification of mutarotase by genetic modification can be performed, as in the aforementioned β-1,4-glucan phosphorylase.

The amount of mutarotase contained in the solution at reaction initiation is typically about 0.01 to 500 U/g β-1,4-glucan, preferably about 0.01 to 100 U/g β-1,4-glucan, more preferably about 0.01 to 50 U/g β-1,4-glucan, particularly preferably about 0.05 to 10 U/g β-1,4-glucan, most preferably about 0.1 to 5 U/g β-1,4-glucan, per β-1,4-glucan in the solution at reaction initiation. When the weight of mutarotase is too large, enzyme denatured during reaction is easily aggregated in some cases. When the amount to be used is too small, the reaction itself occurs, but the yield of glucan is reduced in some cases.

The mutarotase maybe purified, or maybe unpurified. The mutarotase may be immobilized, or may not be immobilized. It is preferable that the mutarotase is immobilized. As the method of immobilization, methods well-known to those skilled in the art such as a carrier binding method (e.g. covalent binding method, ion binding method, or physical adsorbing method), a crosslinking method or an inclusion method (lattice type or a microcapsule type) can be used. It is preferable that the mutarotase is immobilized on a carrier. Further, the mutarotase may be immobilized on the same carrier as that for at least one of the β-1,4-glucan phosphorylase and α-1,4-glucan phosphorylase, or may be immobilized on another carrier. It is preferable that the mutarotase is immobilized on the same carrier as that for both of the β-1,4-glucan phosphorylase and α-1,4-glucan phosphorylase.

(9. Catalase and Peroxidase)

In the production method of the present invention, when glucose oxidase is contained in the solution, it is preferable that catalase or peroxidase is further contained in the solution. By containing catalase or peroxidase in the solution, hydrogen peroxide produced by a reaction catalyzed by glucose oxidase can be converted into oxygen, and oxygen can be recycled. For this reason, by containing glucose oxidase and catalase or peroxidase in the reaction solution, the α-glucose concentration in the reaction solution can be reduced, as a result, the phosphorolysis of cellobiose can be further promoted, and the yield of α-glucan finally obtained can be improved.

The catalase which can be used in the production method of the present invention is an enzyme which catalyzes a reaction degrading hydrogen peroxide into oxygen and water.

Catalase is present in microorganisms, animals and plants. Examples of microorganisms producing catalase include Acetobacter peroxydans, Acholeplasma equifetale, Achole plasma hippikon, Acholeplasma laidlawii, Aspergillus niger, Penicillium janthinellum, Halobacterium halobium, Haloarcula marismortui, Escherichia coli, Mycoplasma arthritidis, Mycoplasma capricolum, Mycobacterium smegmatis, Mycobacterium tuberculosis, Mycoplasma pulmunis, Mycoplasma sp., Bacillus stearothermophilus, Rhodobacter sphaeroides, Lactobacillus plantarum, Thermoleophilum album, Phanerochaete chrysosporium, Saccharomyces cerevisiae, Candida rugosa, Kloeckera sp, Klebsiella pneumoniae, Pseudomona stutzeri and Paracoccus denitrificans. Examples of animals producing catalase include Capra aegagrus hircus, Bos Taurus, Homo sapiens, Rattus norvegicus and Notomastus lobatus (Polychaeta). Examples of plants producing catalase include Gossypium hirsutum, Sinapis alba, Spinacia oleracea, Nicotiana tabacum L., Nicotiana sylvestris, Euglena gracilis (algae) and Pisum sativum. An organism producing catalase is not limited to these.

The catalase which can be used in the present invention is preferably derived from Aspergillus niger, Bovine Liver or Human Erythrocytes, more preferably Aspergillus niger. It is preferable that the catalase used in the present invention has a high optimal reaction temperature. Catalase having a high optimal reaction temperature can be derived, for example, from an extreme thermophilic bacterium.

The peroxidase which can be used in the production method of the present invention is an enzyme which catalyzes oxidation of various organic substances using hydrogen peroxide as a hydrogen acceptor.

Peroxidase is present in microorganisms, animals and plants. Examples of microorganisms producing peroxidase include Pleurotus ostreatus, Halobacterium halobium, Haloarcula marismortui, Coprinus friesii, Phanerochaete chrysosporium, Mycobacterium smegmatis, Mycobacterium tuberculosis, Flavobacterium meningosepticum, Arthromyces ramosus, Phellinus igniarius, Escherichia coli, Thermoleophilum album, Kloeckera sp., Bacillus stearothermophilus, Coprinus cinereus and Coprinus macrorhizus. It should be noted that, in the present specification, a microorganism includes a bacterium and a fungus. Examples of animals producing peroxidase include Homo sapiens, Canis familiaris, Rattus norvegicus, Sus scrofa, and Ovis aries. Examples of plants producing peroxide include horseradish, Armoracia rusticana, Armoracia lapathifolia, Actinidia chinensis, Citrus sinensis, Populus trichocarpa, Nicotiana sylvestris, Picea sitchensis Carr., Picea abies L., Karsten, Petunia hybrida, Carica papaya, Vitis pseudoreticulata, Hordeum vulgare, Brassica rapa, Prunus persica, Vicia faba and Oryza sativa L. An organism producing peroxidase is not limited to these.

The peroxidase which can be used in the present invention is preferably derived from horseradish and Bacillus stearothermophilus, more preferably horseradish. It is preferable that the peroxidase used in the present invention has a high optimal reaction temperature. Peroxidase having a high optimal reaction temperature can be derived, for example, from an extreme thermophilic bacterium.

The catalase or peroxidase which can be used in the present invention can be directly isolated from the aforementioned organisms producing catalase or peroxidase, which are present in the natural world.

The catalase or peroxidase which can be used in the present invention may be isolated from a microorganism (e.g. bacteria, fungi etc.) which has been genetically modified using a gene encoding catalase or peroxidase isolated from these organisms.

Catalase or peroxidase can be obtained from a genetically modified microorganism, as in the aforementioned β-1,4-glucan phosphorylase.

A microorganism (e.g. bacteria, fungi etc.) used in genetic modification can be easily selected, taking various conditions into consideration such as ease of expression of catalase or peroxidase, ease of culturing, proliferation speed, and safety, as in the aforementioned β-1,4-glucan phosphorylase. Since it is preferable that the catalase or peroxidase contains no amylase as a contaminant, it is preferable to use a microorganism (e.g. bacteria, fungi etc.) which does not produce amylase, or expresses amylase only at a low level, for genetic modification. For genetic modification of catalase or peroxidase, it is preferable to use a mesophilic microorganism such as Escherichia coli or Bacillus subtilis. Catalase or peroxidase produced using a microorganism (e.g. bacteria, fungi etc.) which does not produce amylase or expresses amylase only at a low level containing substantially no amylase is preferably used in the method of the present invention.

Production and purification of catalase or peroxidase by genetic modification can be performed, as in the aforementioned β-1,4-glucan phosphorylase.

The amount of catalase or peroxidase contained in the solution at reaction initiation is typically about 0.05 to 1,000 U/g β-1,4-glucan, preferably about 0.1 to 500 U/g β-1,4-glucan, more preferably about 1.0 to 200 U/g β-1,4-glucan, per β-1,4-glucan in the solution at reaction initiation. When the weight of catalase or peroxidase is too large, enzyme denatured during the reaction is easily aggregated in some cases. When the amount to be used is too small, the reaction itself occurs, but the yield of glucan is reduced in some cases.

The catalase or peroxidase may be purified, or may be unpurified. The catalase or peroxidase may be immobilized, or may not be immobilized. It is preferable that the catalase or peroxidase is immobilized. As the method of immobilization, methods well-known to those skilled in the art such as a carrier binding method (e.g. covalent binding method, ion binding method, or physical adsorbing method), a crosslinking method or an inclusion method (lattice type or microcapsule type) can be used. It is preferable that the catalase or peroxidase is immobilized on a carrier. Further, the catalase or peroxidase may be immobilized on the same carrier as that for at least one of the β-1,4-glucan phosphorylase and α-1,4-glucan phosphorylase, or may be immobilized on another carrier. It is preferable that the catalase or peroxidase is immobilized on the same carrier as that for both of the β-1,4-glucan phosphorylase and α-1,4-glucan phosphorylase.

(10. Debranching Enzyme)

In the method of the present invention, when a branch is generated in a product such as when a starting material containing an α-1,6-glucoside bond is used, a debranching enzyme can be used, as necessary.

Debranching enzymes which can be used in the present invention are enzymes which can cut an α-1,6-glucoside bond. The debranching enzymes are classified into two groups, one of which is isoamylase (EC 3.2.1.68) which well acts on both amylopectin and glycogen, and the other of which is α-dextrinendo-1,6-α-glucosidase (also referred to as pullulanase) (EC 3.2.1.41) which acts on amylopectin, glycogen and pullulan.

Debranching enzymes are present in microorganisms and plants. Examples of microorganisms producing a debranching enzyme include Saccharomyces cerevisiae, Chlamydomonas sp., Bacillus brevis, Bacillus acidopullulyticus, Bacillus macerans, Bacillus stearothermophilus, Bacillus circulans, Thermus aquaticus, Klebsiella pneumoniae, Thermoactinomyces thalpophilus, Thermoanaerobacter ethanolicus and Pseudomonas amyloderamosa. Examples of plants producing a debranching enzyme include potato, sweet potato, corn, rice, wheat, barley, rye, and beet. An organism producing a debranching enzyme is not limited to these.

A debranching enzyme which can be used in the present invention is preferably derived from Klebsiella pneumoniae, Bacillus brevis, Bacillus acidopullulyticus, or Pseudomonas amyloderamosa, more preferably Klebsiella pneumoniae, or Pseudomonas amyloderamosa. It is preferable that a debranching enzyme used in the present invention has a high optimal reaction temperature. A debranching enzyme having a high optimal reaction temperature can be derived, for example, from an extreme thermophilic bacterium.

A debranching enzyme which can be used in the present invention can be directly isolated from the aforementioned microorganisms and plants producing debranching enzymes, which are present in the natural world.

A debranching enzyme which can be used in the present invention may be isolated from a microorganism (e.g. bacteria, fungi etc.) which has been genetically modified using a gene encoding a debranching enzyme isolated from these microorganisms and plants.

A debranching enzyme can be obtained from a genetically modified microorganism, as in the aforementioned β-1,4-glucan phosphorylase.

A microorganism (e.g. bacteria, fungi etc.) used in genetic modification can be easily selected, taking various conditions into consideration, such as ease of expression of a debranching enzyme, ease of culturing, proliferation speed, and safety, as in the aforementioned β-1,4-glucan phosphorylase. Since it is preferable that a debranching enzyme contain no amylase as a contaminant, it is preferable to use a microorganism (e.g. bacteria, fungi etc.) which does not produce amylase or expresses amylase only at a low level for genetic modification. For genetic modification of a debranching enzyme, it is preferable to use a mesophilic microorganism such as Escherichia coli or Bacillus subtilis. A debranching enzyme produced using a microorganism (e.g. bacteria, fungi etc.) which does not produce amylase or expresses amylase only at a low level containing substantially no amylase is preferably used in the method of the present invention.

Production and purification of a debranching enzyme by genetic modification can be performed, as in the aforementioned β-1,4-glucan phosphorylase.

The amount of a debranching enzyme contained in the solution at reaction initiation is typically about 0.05 to 1,000 U/g β-1,4-glucan, preferably about 0.1 to 500 U/g β-1,4-glucan, more preferably about 0.5 to 100 U/g β-1,4-glucan, per β-1,4-glucan in the solution at reaction initiation. When the weight of a debranching enzyme is too large, enzyme denatured during the reaction is easily aggregated in some cases. When an amount to be used is too small, the reaction itself occurs, but the yield of glucan is reduced in some cases.

A debranching enzyme may be purified or may be unpurified. A debranching may be immobilized, or may not be immobilized. It is preferable that a debranching enzyme is immobilized. As the method of immobilization, methods well-known to those skilled in the art such as a carrier binding method (e.g. covalent binding method, ion binding method, or physical adsorbing method), a crosslinking method or an inclusion method (lattice type or microcapsule type) can be used. It is preferable that a debranching enzyme is immobilized on a carrier. Further, a debranching enzyme may be immobilized on the same carrier as that for at least one of the β-1,4-glucan phosphorylase and α-1,4-glucan phosphorylase, or may be immobilized on other carrier. It is preferable that a debranching enzyme is immobilized on the same carrier as that for both of the β-1,4-glucan phosphorylase and α-1,4-glucan phosphorylase.

(11. Branching Enzyme (EC 2.4.1.18))

In the method of the present invention, when it is desired that a branch is generated in a product, a branching enzyme may be used, as necessary.

A branching enzyme which can be used in the present invention is an enzyme which transfers a part of an α-1,4-glucan chain to position 6 of a certain glucose residue within this α-1,4-glucan chain to make a branch. Branching enzymes are also called 1,4-α-glucan branching enzymes, a branch making enzymes or Q enzymes.

Branching enzymes are present in microorganisms, animals and plants. Examples of microorganisms producing branching enzymes include Bacillus stearothermophilus, Bacillus subtilis, Bacillus caldolyticus, Bacillus licheniformis, Bacillus amyloliquiefaciens, Bacillus coagulans, Bacillus caldovelox, Bacillus thermocatenulatus, Bacillus smithii, Bacillus megaterium, Bacillus brevis, Alkalophillic Bacillus sp., Streptomyces coelicolor, Aquifex aeolicus, Synechosystis sp., E. coli, Agrobacterium tumefaciens, Thermus aquaticus, Rhodothermus obamensis, Neurospora crassa and yeast. Examples of animals producing branching enzymes include mammals such as human, rabbit, rat and pig. Examples of plants producing branching enzymes include algae; tubers such as potato, sweet potato, yam, and cassava; vegetables such as spinach; cereals such as corn, rice, wheat, barley, rye, and foxtail millet; beans such as pea, soybean, adzuki bean, and mottled kidney bean; and the like. An organism producing branching enzymes is not limited to these.

A branching enzyme which can be used in the present invention is preferably derived from potato, Bacillus stearothermophilus, or Aquifex aeolicus, more preferably Bacillus stearothermophilus, or Aquifex aeolicus. It is preferable that a branching enzyme used in the present invention has a high optimal reaction temperature. A branching enzyme having a high optimal reaction temperature can be derived, for example, from an extreme thermophilic bacterium.

A branching enzyme which can be used in the present invention can be directly isolated from the aforementioned microorganisms, animals and plants producing a branching enzyme, which are present in the natural world.

A branching enzyme which can be used in the present invention may be isolated from a microorganism (e.g. bacteria, fungi etc.) which has been genetically modified using a gene encoding a branching enzyme isolated from these microorganisms, animals and plants.

A branching enzyme can be obtained from a genetically modified microorganism, as in the aforementioned β-1,4-glucan phosphorylase.

A microorganism (e.g. bacteria, fungi etc.) used in genetic modification can be easily selected, taking various conditions into consideration such as ease of expression of a branching enzyme, ease of culturing, proliferation speed, and safety, as in the aforementioned β-1,4-glucan phosphorylase. Since it is preferable that a branching enzyme contain no amylase as a contaminant, it is preferable to use a microorganism (e.g. bacteria, fungi etc.) which does not produce amylase or expresses amylase only at a low level, for genetic modification. For genetic modification of a branching enzyme, it is preferable to use a mesophilic microorganism such as Escherichia coli or Bacillus subtilis. A branching enzyme produced using a microorganism (e.g. bacteria, fungi etc.) which does not produce amylase or expresses amylase only at a low level containing substantially no amylase is preferably used in the method of the present invention.

Production and purification of a branching enzyme by genetic modification can be performed, as in the aforementioned β-1,4-glucan phosphorylase.

The amount of a branching enzyme contained in the solution at reaction initiation is typically about 10 to 100,000 U/g β-1,4-glucan, preferably about 100 to 50,000 U/g β-1,4-glucan, more preferably about 1,000 to 10,000 U/g β-1,4-glucan, per β-1,4-glucan in the solution at reaction initiation. When the weight of a branching enzyme is too large, enzyme denatured in a reaction is easily aggregated in some cases. When the amount to be used is too small, the reaction itself occurs, but the yield of glucan is reduced in some cases.

A branching enzyme may be purified, or may be unpurified. A branching enzyme may be immobilized, or may not be immobilized. It is preferable that a branching enzyme is immobilized. As the method of immobilization, methods well-known to those skilled in the art such as a carrier binding method (e.g. covalent binding method, ion binding method, or physical adsorbing method), a crosslinking method or an inclusion method (lattice type or microcapsule type) can be used. It is preferable that a branching enzyme is immobilized on a carrier. Further, a branching enzyme may be immobilized on the same carrier as that for at least one of the β-1,4-glucan phosphorylase and α-1,4-glucan phosphorylase, or may be immobilized on other carrier. It is preferable that a branching enzyme is immobilized on the same carrier as that for both of the β-1,4-glucan phosphorylase and α-1,4-glucan phosphorylase.

(12. 4-α-glucanotransferase (EC 2.4.1.25))

In the method of the present invention, when a cyclic structure is generated as a product, 4-α-glucanotransferase can be used, as necessary.

The 4-α-glucanotransferase which can be used in the present invention is also called disproportionating enzyme, D-enzyme, or amylomaltase, and is an enzyme which can catalyze a saccharide transferring reaction (disproportionation reaction) of maltooligosaccharide. 4-α-glucanotransferase is a enzyme which transfers a glucosyl group, or a maltosyl or maltooligosyl unit from a non-reducing terminal of a donor molecule to a non-reducing terminal of an acceptor molecule. Therefore, the enzymatic reaction results in disproportionation of the degree of polymerization of the first given maltooligosaccharide. When a donor molecule and an acceptor molecule are the same, intramolecular transfer occurs and, as a result, a product having a cyclic structure is obtained.

4-α-glucanotransferase is present in microorganisms and plants. Examples of microorganisms producing 4-α-glucanotransferase include Aquifex aeolicus, Streptococcus pneumoniae, Clostridium butylicum, Deinococcus radiodurans, Haemophilus influenzae, Mycobacterium tuberculosis, Thermococcus litralis, Thermotoga maritima, Thermotoga neapolitana, Chlamydia psittaci, Pyrococcus sp., Dictyoglomus thermophilum, Borrelia burgdorferi, Synechosystis sp., E. coli and Thermus aquaticus. Examples of plants producing 4-α-glucanotransferase include tubers such as potato, sweet potato, yam, and cassava; cereals such as corn, rice, and wheat; beans such as pea, and soybean; and the like. An organism producing 4-α-glucanotransferase is not limited to these.

The 4-α-glucanotransferase which can be used in the present invention is preferably derived from potato, Thermus aquaticus, or Thermococcus litralis, more preferably potato, or Thermus aquaticus. It is preferable that the 4-α-glucanotransferase used in the present invention has a high optimal reaction temperature. 4-α-glucanotransferase having a high optimal reaction temperature can be derived, for example, from an extreme thermophilic bacterium.

The 4-α-glucanotransferase which can be used in the present invention can be directly isolated from the aforementioned microorganisms and plants producing 4-α-glucanotransferase, which are present in the natural world.

The 4-α-glucanotransferase which can be used in the present invention may be isolated from a microorganism (e.g. bacteria, fungi etc.) which has been genetically modified using a gene encoding 4-α-glucanotransferase isolated from these microorganisms and plants.

4-α-glucanotransferase can be obtained from a genetically modified microorganism, as in the aforementioned β-1,4-glucan phosphorylase.

A microorganism (e.g. bacteria, fungi etc.) used in genetic modification can be easily selected, taking various conditions into consideration such as ease of expression of 4-α-glucanotransferase, ease of culturing, proliferation speed, and safety, as in the aforementioned β-1,4-glucan phosphorylase. Since it is preferable that 4-α-glucanotransferase contain no amylase as a contaminant, it is preferable to use a microorganism (e.g. bacteria, fungi etc.) which does not produce amylase or expresses amylase only at a low level for genetic modification. For genetic modification of 4-α-glucanotransferase, it is preferable to use a mesophilic microorganism such as Escherichia coli or Bacillus subtilis. 4-α-glucanotransferase produced using a microorganism (e.g. bacteria, fungi etc.) which does not produce amylase or expresses amylase only at a low level containing substantially no amylase is preferably used in the method of the present invention.

Production and purification of 4-α-glucanotransferase by genetic modification can be performed, as in the aforementioned β-1,4-glucan phosphorylase.

The amount of 4-α-glucanotransferase contained in the solution at reaction initiation is typically about 0.05 to 1,000 U/g β-1,4-glucan, preferably about 0.1 to 500 U/g β-1,4-glucan, more preferably about 0.5 to 100 U/g β-1,4-glucan, per β-1,4-glucan in the solution at reaction initiation. When the weight of 4-α-glucanotransferase is too large, enzyme denatured during the reaction is easily aggregated in some cases. When the amount to be used is too small, the reaction itself occurs, but the yield of glucan is reduced in some cases.

The 4-α-glucanotransferase may be purified, or may be unpurified. The 4-α-glucanotransferase may be immobilized, or may not be immobilized. It is preferable that the 4-α-glucanotransferase is immobilized. As the method of immobilization, methods well-known to those skilled in the art such as a carrier binding method (e.g. covalent binding method, ion binding method, or physical adsorbing method), a crosslinking method or an inclusion method (lattice type or microcapsule type) can be used. It is preferable that the 4-α-glucanotransferase is immobilized on a carrier. Further, the 4-α-glucanotransferase may be immobilized on the same carrier as that for at least one of the β-1,4-glucan phosphorylase and the α-1,4-glucan phosphorylase, or may be immobilized on other carrier. It is preferable that 4-α-glucanotransferase is immobilized on the same carrier as that for both of the β-1,4-glucan phosphorylase and α-1,4-glucan phosphorylase.

(13. Glycogen Debranching Enzyme (EC. 2.4.1.25/EC. 3.2.1.33))

In the method of the present invention, when a cyclic structure is generated in a product, a glycogen debranching enzyme can be used, as necessary.

A glycogen debranching enzyme which can be used in the present invention is an enzyme having the two kinds of activities of an α-1,6-glucosidase activity and a 4-α-glucanotransferase activity. Through the 4-α-glucanotransferase activity possessed by a glycogen debranching enzyme, a product having a cyclic structure is obtained.

Glycogen debranching enzymes are present in microorganisms and animals. Examples of microorganisms producing glycogen debranching enzymes include yeast and the like. Examples of animals producing glycogen debranching enzymes include mammals such as human, rabbit, rat and pig. An organism producing a glycogen debranching enzyme is not limited to these.

It is preferable that a glycogen debranching enzyme which can be used in the present invention is derived from yeast. It is preferable that a glycogen debranching enzyme used in the present invention has a high optimal reaction temperature. A glycogen debranching enzyme having a high optimal reaction temperature is obtained, for example, by introducing a modification to an enzyme which can act at an intermediate temperature, by protein engineering techniques.

A glycogen debranching enzyme which can be used in the present invention can be directly isolated from the aforementioned microorganisms and animals producing glycogen debranching enzymes, which are present in the natural world.

A glycogen debranching enzyme which can be used in the present invention may be isolated from a microorganism (e.g. bacteria, fungi etc.) which has been genetically modified using a gene encoding a glycogen debranching enzyme isolated from these microorganisms and animals.

A glycogen debranching enzyme can be obtained from a genetically modified microorganism, as in the aforementioned β-1,4-glucan phosphorylase.

A microorganism (e.g. bacteria, fungi etc.) used in genetic modification can be easily selected, taking various conditions into consideration such as ease of expression of a glycogen debranching enzyme, ease of culturing, proliferation speed, and safety, as in the aforementioned β-1,4-glucan phosphorylase. Since it is preferable that a glycogen debranching enzyme contain no amylase as a contaminant, it is preferable to use a microorganism (e.g. bacteria, fungi etc.) which does not produce amylase or expresses amylase only at a low level for genetic modification. For genetic modification of a glycogen debranching enzyme, it is preferable to use a mesophilic microorganism such as Escherichia coli or Bacillus subtilis. A glycogen debranching enzyme produced using a microorganism (e.g. bacteria, fungi etc.) which does not produce amylase or expresses amylase only at a low level containing substantially no amylase is preferably used in the method of the present invention.

Production and purification of a glycogen debranching enzyme by genetic modification can be performed, as in the aforementioned β-1,4-glucan phosphorylase.

The amount of a glycogen debranching enzyme contained in the solution at reaction initiation is typically about 0.01 to 5,000 U/g β-1,4-glucan, preferably about 0.1 to 1,000 U/g β-1,4-glucan, more preferably about 1 to 500 U/g β-1,4-glucan, per β-1,4-glucan in the solution at reaction initiation. When the weight of a glycogen debranching enzyme is too large, enzyme denatured during the reaction is easily aggregated in some cases. When the amount to be used is too small, the reaction itself occurs, but the yield of glucan is reduced in some cases.

A glycogen debranching enzyme may be purified, or may be unpurified. A glycogen debranching enzyme may be immobilized, or may not be immobilized. It is preferable that a glycogen debranching enzyme is immobilized. As the method of immobilization, methods well-known to those skilled in the art such as a carrier binding method (e.g. covalent binding method, ion binding method, or physical adsorbing method), a crosslinking method or an inclusion method (lattice type or microcapsule type) can be used. It is preferable that a glycogen debranching enzyme is immobilized on a carrier. Further, a glycogen debranching enzyme may be immobilized on the same carrier as that for at least one of the β-1,4-glucan phosphorylase and the α-1,4-glucan phosphorylase, or may be immobilized on other carrier. It is preferable that a glycogen debranching enzyme is immobilized on the same carrier as that for both of the β-1,4-glucan phosphorylase and α-1,4-glucan phosphorylase.

(14. Solvent)

A solvent used in the method of the present invention can be any solvent as long as it is a solvent which does not deteriorate the enzymatic activities of the β-1,4-glucan phosphorylase and α-1,4-glucan phosphorylase.

It should be noted that, as long as the reaction producing glucan continues to progress, it is not necessary that the solvent completely dissolves the materials used in the method of the present invention. For example, when an enzyme is carried on a solid carrier, it is not necessary that the enzyme is dissolved in a solvent. Further, it is not necessary that not all of reaction materials, such as β-1,4-glucan, are dissolved, and a part of the materials may be dissolved to such an extent that the reaction can progress.

A typical solvent is water. A solvent may be water in a cell lysate, accompanied with β-1,4-glucan phosphorylase or α-1,4-glucan phosphorylase upon the preparation of the aforementioned β-1,4-glucan phosphorylase or α-1,4-glucan phosphorylase.

Water may be any of soft water, intermediate water and hard water. Hard water refers to water having a hardness of 20° or higher, intermediate water refers to water having a hardness of not lower than 10° and lower than 20°, and soft water refers to water having a hardness lower than 10°. Water is preferably soft water or intermediate water, more preferably soft water.

(15. Other Components)

The solution containing the β-1,4-glucan, the primer, the inorganic phosphoric acid or glucose-1-phosphate, the β-1,4-glucan phosphorylase and the α-1,4-glucan phosphorylase may contain any other substance as long as it does not prevent interaction between the β-1,4-glucan phosphorylase and the β-1,4-glucan and interaction between the α-1,4-glucan phosphorylase and the primer. Examples of such substances include buffers, components of a microorganism (e.g. bacteria, fungi etc.) producing β-1,4-glucan phosphorylase, components of a microorganism (e.g. bacteria, fungi etc.) producing α-1,4-glucan phosphorylase, salts, a medium component and the like.

<Production of an α-Glucan>

The α-glucan of the present invention is produced by reacting a solution containing a β-1,4-glucan, a primer, inorganic phosphoric acid or glucose-1-phosphate, β-1,4-glucan phosphorylase, and α-1,4-glucan phosphorylase.

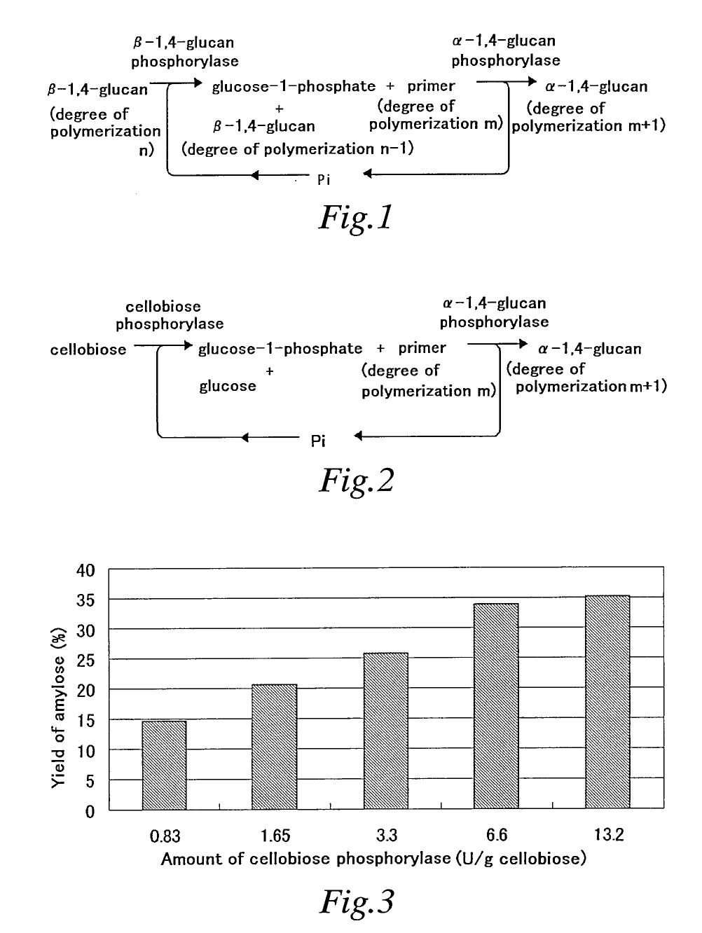

FIG. 2 shows a schematic of the reaction generated in the production method of the present invention. Glucose-1-phosphate and a β-1,4-glucan (degree of polymerization n−1) are produced from a β-1,4-glucan (degree of polymerization n) and inorganic phosphoric acid using β-1,4-glucan phosphorylase. Produced glucose-1-phosphate (and glucose-1-phosphate which has been added to the solution) are immediately transferred to an appropriate primer (degree of polymerization m) by an α-1,4-bond by α-1,4-glucan phosphorylase, and extended as an α-glucan chain (degree of polymerization m+1). Further, there is a mechanism that the inorganic phosphoric acid produced thereupon is recycled to a reaction of β-1,4-glucan phosphorylase again.

In addition, a schematic of the reaction generated in the production method of the present invention when the initial β-1,4-glucan is cellobiose, and the β-1,4-glucan phosphorylase is cellobiose phosphorylase is shown in FIG. 2. Glucose-1-phosphate and glucose are produced from cellobiose (degree of polymerization 2) and inorganic phosphoric acid using cellobiose phosphorylase. Produced glucose-1-phosphate (and glucose-1-phosphate which has been added to the solution) are immediately transferred to an appropriate primer (degree of polymerization m) by an α-1,4-linkage with α-1,4-glucan phosphorylase, and the α-glucan chain (degree of polymerization m+1) is extended. In addition, inorganic phosphoric acid produced thereupon is recycled to the reaction of β-1,4-glucan phosphorylase again.

In the production method of the present invention, for example, a solution is firstly prepared. The solution can be prepared, for example, by adding solid β-1,4-glucan, the primer, inorganic phosphoric acid or glucose-1-phosphate, the β-1,4-glucan phosphorylase, and the α-1,4-glucan phosphorylase to an appropriate solvent. Alternatively, the solution may be prepared by mixing solutions, each containing the β-1,4-glucan, the primer, a source of phosphoric acid such as inorganic phosphoric acid or glucose-1-phosphate, the β-1,4-glucan phosphorylase, or the α-1,4-glucan phosphorylase. Alternatively, the solution may be prepared by mixing a solution containing some components of the β-1,4-glucan, the primer, a source of phosphoric acid such as inorganic phosphoric acid or glucose-1-phosphate, the β-1,4-glucan phosphorylase, and the α-1,4-glucan phosphorylase, with other components in solid form. Any buffer may be added as necessary to the solution used in the production method of the present invention for the purpose of adjusting pH, as long as the enzymatic reaction is not inhibited. The pH of this solution can be any pH as long as the enzymatic reaction is not excessively inhibited. The pH value is preferably about 6 to about 8, more preferably about 6.5 to about 7.5. The pH can be appropriately set in conformity with the optimal pH of the enzyme used in the reaction. The salt concentration of the solution may be any salt concentration as long as the enzymatic reaction is not excessively inhibited. The salt concentration is preferably 1.0 mM to 50 mM, more preferably 5 mM to 30 mM.

When the β-1,4-glucan is cellobiose, and the β-1,4-glucan phosphorylase is cellobiose phosphorylase, for example, glucose isomerase or glucose oxidase (and mutarotase) may be further added to this solution in order to remove, from the solution, glucose produced upon production of α-glucan. Further, catalase or peroxidase may be added to the solution. Alternatively, a microorganism which removes glucose from the solution by metabolizing glucose, such as yeast, may added. Alternatively, a glucose-specific adsorbing resin may be added. A method of adding an enzyme or a microorganism is preferable, since this method can remove glucose while the reaction continuously progresses. It should be noted that “remove”, as used herein, includes reduction in the amount of glucose in the reaction solution, and elimination of the presence of glucose.

In addition, an enzyme selected from the group consisting of debranching enzymes, branching enzymes, 4-α-glucanotransferase and glycogen debranching enzymes may be added to this solution, as necessary. These enzymes may be added at initiation of the α-glucan synthesizing reaction, may be added during the reaction, or may be added after completion of the reaction.

Then, the solution is reacted by heating the solution by methods known in the art, as necessary. The temperature of the solution is any temperature as long as the effect of the present invention is obtained, and is a temperature at which the added enzyme exhibits its activity. For example, by using a thermostable enzyme and adjusting the reaction temperature to an optimal temperature for the thermostable enzyme, activity of any contaminated enzyme other than the added thermostable enzyme can be suppressed. It is preferable that the temperature of the solution in this reaction step is such a temperature that activity which is about 50% or more, more preferably about 80% or more of activity of at least one of, preferably both of, the β-1,4-glucan phosphorylase and the glucan phosphorylase contained in this solution before the reaction remain(s) after a predetermined reaction time. This temperature is preferably about 30° C. to about 70° C., more preferably about 35° C. to about 60° C.

The reaction time can be set at any term taking the reaction temperature, the molecular weight of a glucan produced by the reaction, and the remaining activity of the enzyme into consideration. The reaction time is typically about 1 hour to about 100 hours, more preferably about 1 hour to about 72 hours, further more preferably about 2 hours to about 36 hours, most preferably about 2 hours to about 24 hours.

Heating may be performed using any means, and it is preferable to perform heating while stirring so that the heat is transmitted uniformly to the whole solution. The solution is stirred, for example, by placement into a stainless reaction tank equipped with a warm water jacket and a stirring device.

In the method of the present invention, at the stage at which the reaction has progressed to some extent, at least one of the β-1,4-glucan, the β-1,4-glucan phosphorylase and the α-1,4-glucan phosphorylase may be additionally added to the reaction solution.

When the β-1,4-glucan is cellobiose, and the β-1,4-glucan phosphorylase is cellobiose phosphorylase, as described above, it is preferable to perform a step of adding an enzyme such as glucose isomerase to remove glucose produced as a byproduct at the same time as production of the α-glucan, simultaneously with the production step. On the other hand, the step of removing glucose may be performed at a different time from the step of producing α-glucan. For example, in the method of the present invention, at the stage at which the reaction has progressed to some extent, in order to remove glucose produced by the reaction, the solution is treated by a method for physically removing glucose such as chromatography fractionation and a membrane fractionating method, and the reaction may progress again thereafter. A method for physically removing glucose may be performed once, or may be performed two or more times. When the method is performed two or more times, for example, after the reaction has proceeded for 2 hours, glucose may be removed and, then, the reaction may be performed for 2 hours again, glucose may be removed, and then the reaction may be performed for 2 hours again.

A solution containing α-glucan is produced in this manner.

After completion of the reaction, the reaction is heated, as necessary, for example, at 100° C. for 10 minutes, to inactivate the enzymes in the solution. Alternatively, a post-step may be performed without performing treatment for inactivating the enzymes. A solution may be stored as is, or may be treated for isolating the produced glucan.

<Purification Method>

The produced α-glucan can be purified as necessary. An example of an impurity which is removed by purification is glucose. As an example of a method of purifying α-glucan, there are methods of using an organic solvent (T. J. Schoch et al., J. American Chemical Society, 64, 2957 (1942)) and methods not using an organic solvent.

Examples of an organic solvent which can be used in purification using an organic solvent include acetone, n-amyl alcohol, pentazole, n-propyl alcohol, n-hexyl alcohol, 2-ethyl-1-butanol, 2-ethyl-1-hexanol, lauryl alcohol, cyclohexanol, n-butyl alcohol, 3-pentanol, 4-methyl-2-pentanol, d,l-borneol, α-terpineol, isobutyl alcohol, sec-butyl alcohol, 2-methyl-1-butanol, isoamyl alcohol, tert-amyl alcohol, menthol, methanol, ethanol and ether.

Examples of a purification method not using an organic solvent will be shown below.

(1) A method of, after the α-glucan producing reaction, cooling the reaction solution to precipitate α-glucan, and purifying the precipitated α-glucan by a general solid-liquid separating method such as membrane fractionation, filtration, and centrifugation;

(2) a method of, during the α-glucan producing reaction or after the reaction of producing α-glucan, cooling the reaction solution to gel the α-glucan, recovering the gelled α-glucan, and removing glucose from the gelled α-glucan by a procedure such as washing with water, freeze-thaw and filtration; and

(3) a method of, after the reaction of producing α-glucan, subjecting the reaction mixture to membrane fractionation using ultrafiltration or chromatography without precipitating the α-glucan dissolved in water, to remove glucose.

Examples of ultrafiltration membranes which can be used in purification include an ultrafiltration membrane (UF membrane unit manufactured by Daicel) with a fractionating molecular weight of about 1,000 to about 100,000, preferably about 5,000 to about 50,000, more preferably about 10,000 to about 30,000.

Examples of a carrier which can be used in chromatography include a carrier for gel filtration chromatography, a carrier for ligand exchange chromatography, a carrier for ion exchange chromatography and a carrier for hydrophobic chromatography.

### EXAMPLE 1

The present invention will be explained in more detail below by way of the following Examples. The present invention is not limited to the following Examples.

(1. Measuring Method and Calculating Method)

The activities of various enzymes in the present invention and the yield of the resulting α-glucan were measured by the following measuring method.

(1.1 A Method for Measuring Activity of Cellobiose Phosphorylase)

30 μl of a 40 mM aqueous cellobiose solution and 30 μl of a 40 mM aqueous sodium phosphate solution (pH 7.5) are mixed, 60 μl of an appropriately diluted enzyme solution (sample) is further added, and reaction is initiated in 120 μl of the mixture. After this mixture is incubated at 37° C. for 10 minutes in order for the reaction to proceed, the mixture is retained at 100° C. for 10 minutes to inactivate the enzyme. Subsequently, 780 μl of a 1M Tris-HCl buffer (pH 7.0) and 120 μl of a coloring reagent (glucose AR-II coloring reagent (manufactured by Wako Pure Chemical Industries, Ltd.)) are added to this mixture, mixed, and the absorbance at 505 nm is measured. The absorbance is similarly measured using an aqueous glucose solution having a known concentration, and a standard curve is produced. The absorbance obtained by the sample is fitted to this standard curve to obtain the glucose amount in the sample. One unit of cellobiose phosphorylase is defined as the amount of the enzyme producing 1 μmol of glucose in one minute from 20 mM cellobiose using the aforementioned method.

(1.2 A Method for Measuring Activity of α-1,4-Glucan Phosphorylase)

50 μl of a 4% aqueous cluster dextrin solution and 50 μl of a 50 mM aqueous sodium glucose-1-phosphate solution are mixed, 100 μl of an appropriately diluted enzyme solution is further added, and the reaction is initiated in 200 μl of the mixture. After this mixture is incubated at 37° C. for 15 minutes in order for the reaction to proceed, 800 μl of a molybdenum reagent (15 mM ammonium molybdate, 100 mM zinc acetate) is added to the mixture to stop the reaction. Subsequently, 200 μl of 568 mM ascorbic acid (pH 5.0) is added, and this is stirred to obtain a reaction system. After this reaction system is retained at 30° C. for 20 minutes, the absorbance at 850 nm is measured using a spectrophotometer. The absorbance is similarly measured using inorganic phosphoric acid having a known concentration, and a standard curve is produced. The absorbance obtained by the sample is fitted to this standard curve to obtain the amount of inorganic phosphoric acid in the sample. The activity of producing 1 μmol inorganic phosphoric acid in one minute by this method is defined as one unit of α-1,4-glucan phosphorylase.

(1.3 Method of Calculating Yield of Resulting α-Glucan)

The yield of α-glucan by the production method of the present invention was calculated based on the fact that the mole number of a glucose residue taken into the resulting α-glucan corresponds to a % of the mole number of the initially added cellobiose. Ethanol was added to the solution after completion of the reaction to a final concentration of 50%, to precipitate the α-glucan, the supernatant was discarded, the α-glucan was further washed with an appropriate amount of 50% ethanol two times, dried, and dissolved in an appropriate amount of water, and then the concentration of glucose was measured by the phenol-sulfuric acid method, thereby, the amount (mole number) of the α-glucan was calculated. The yield was calculated by dividing this yield amount (mole number) by the mole number of cellobiose, and multiplying this with 100. This calculating equation is shown in the following equation.
(α-glucan yield (%))=(α-glucan (mM glucose equivalent))/(initial cellobiose (mM))×100 (Equation 2)

(1.4 A Method for Measuring Weight Average Molecular Weight of α-Glucan)

An α-glucan synthesized by the present invention was completely dissolved in 1N sodium hydroxide, neutralized with an appropriate amount of hydrochloric acid, and an about 300 μg aliquot of the α-glucan was subjected to gel filtration chromatography using a differential refractometer and a multiangular light scatter detector together to obtain the average molecular weight.

More particularly, Shodex SB806M-HQ (manufactured by SHOWA DENKO K.K.) was used as the column and a multiangular light scatter detector (DAWN-DSP, manufactured by Wyatt Technology) and a differential refractometer (Shodex RI-71, manufactured by SHOWA DENKO K.K.) were used as the detector by connecting them in this order. The column was maintained at 40° C. and, as an eluent, a 0.1 M sodium nitrate solution was used at a flow rate of 1 mM/min. The resulting signal was collected using a data analyzing software (trade name: ASTRA, manufactured by Wyatt Technology), and this was analyzed using the same software, and a weight average molecular weight was thereby obtained.

(2. Preparation of Enzyme)

Various enzymes used in Examples of the present invention were prepared by the following methods.

(2.1 Method for Preparing Recombinant Cellobiose Phosphorylase)

Chromosome genes of Clostridium thermocellum were extracted, and this was used as a template. By performing PCR using the following two kinds of synthetic DNA primers: synthetic DNA primer 1: 5′aaactctagaaataattttgtttaactttaagaaggagatataccatggagtt cggtttttttgatgat 3′ (SEQ ID NO: 1) and synthetic DNA primer 2: 5′aaactcgagaattacttcaactttgtgagtcttt 3′ (SEQ ID NO: 2), under the heating conditions of 30 cycles of 1 minute at 98° C., 1 minute at 55° C., and 3 minutes at 68° C. in this order, a region containing the CBP gene was amplified. The amplified gene together with a selectable marker gene Km^{r} was incorporated into an expression vector pET28a (manufactured by STRATAGENE) to obtain a plasmid pET28α-CBP1. In this plasmid, the cellobiose phosphorylase gene was operably linked under the control of an isopropyl-β-D-thiogalactopyranoside (IPTG) inducible promoter.

This plasmid was introduced into Escherichia coli BL21 (DE3) pLysS (manufactured by STRATAGENE) by the competent cell method. This Escherichia coli was plated on a plate containing LB medium (1% tryptone (manufactured by Difco), 0.5% yeast extract (manufactured by Difco), 1% sodium chloride, 1.5% agar)) containing the antibiotic kanamycin, and this was cultured at 37° C. overnight. Escherichia coli grown on this plate was selected to obtain Escherichia coli in which the cellobiose phosphorylase gene derived from Clostridium thermocellum had been introduced.

It was confirmed by analyzing the sequence of the introduced gene that the resulting Escherichia coli contains the cellobiose phosphorylase gene. In addition, it was confirmed by activity measurement that the resulting Escherichia coli expresses the cellobiose phosphorylase.

This Escherichia coli was inoculated on 1 liter of LB medium (1% tryptone, 0.5% yeast extract (both manufactured by Dif co), 1% sodium chloride) containing the antibiotic kanamycin, and this was cultured at 37° C. for 3 hours with shaking at 120 rpm. Thereafter, IPTG was added to this medium to 1.0 mM, and this was cultured at 37° C. for further 8 hours with shaking. Then, this culture was centrifuged at 5,000 rpm for 5 minutes to collect the Escherichia coli cells. The resulting cells were suspended in 50 ml of 50 mM phosphate buffer (pH 7.5) containing 1.4 mM 2-mercaptoethanol and, then, this was crushed by sonication to obtain 50 ml of crushed cell liquid. This crushed liquid contained 132 U/ml cellobiose phosphorylase.

This crushed cell liquid was heated at 55° C. for 20 minutes. After heating, this was centrifuged at 8,500 rpm for 20 minutes to remove insoluble proteins and the like to obtain the supernatant. The resulting supernatant was applied to a pre-equilibrated His-Tag adsorption resin Ni-NTA agarose (manufactured by QIAGEN), allowing cellobiose phosphorylase to be adsorbed onto this resin. This resin was washed with a buffer containing 300 mM sodium chloride,

TABLE 1

| | Composition | | | | | |
|---|---|---|---|---|---|---|
| | | | concentration | | | |
| | Concentration | Concentration | of | | | Weight |
| | of primer | of | phosphoric | CBP | GP | average |
| | (G4*¹) | cellobiose | acid*² | (U/g | (U/g | molecular |
| No. | (μM) | (%) | (mM) | cellobiose) | cellobiose) | weight |
| Example | 6,000 | 3 | 30 | 6.6 | 50 | 14,560 |
| 1-1 | | | | | | |
| Example | 3,000 | 3 | 30 | 6.6 | 50 | 22,920 |
| 1-2 | | | | | | |
| Example | 100 | 3 | 30 | 6.6 | 50 | 51,120 |
| 1-3 | | | | | | |
| Example | 75 | 3 | 30 | 6.6 | 50 | 136,100 |
| 1-4 | | | | | | |
| Example | 9.4 | 3 | 30 | 6.6 | 50 | 306,500 |
| 1-5 | | | | | | |
| Example | 2.4 | 3 | 30 | 6.6 | 50 | 461,200 |
| 1-6 | | | | | | |

*¹G4: maltotetraose
*²Phosphoric acid was added as a potassium dihydrogen phosphate-disodium hydrogen phosphate buffer. The pH of the phosphate buffer is 7.0.

After reaction, the weight average molecular weights of synthesized amyloses were determined according to the aforementioned 1.4. Results are shown in Table 1.

As a result, by performing the reaction of acting cellobiose phosphorylase (CBP) on cellobiose in the presence of phosphoric acid to produce glucose-1-phosphate and glucose, and the reaction of acting glucan phosphorylase (GP) on glucose-1-phosphate in the presence of a primer to transfer a glucose residue to the primer in the same solution, amylose could be produced. In addition, it was confirmed that, by changing the concentration of primer in the reaction solution, the degree of polymerization of the synthesized amylose can be freely controlled, that is, when high molecular weight 20 mM imidazole and 1.4 mM 2-mercaptoethanol to remove impurities. Subsequently, proteins were eluted with a buffer containing 300 mM sodium chloride, 150 mM imidazole and 1.4 mM 2-mercaptoethanol, to obtain the recombinant cellobiose phosphorylase enzyme solution.

(2.2 Method for Preparing Recombinant Potato α-1,4-Glucan Phosphorylase)

A potato α-1,4-glucan phosphorylase gene (Nakano et al., Journal of Biochemistry (Tokyo) 106 (1989) 691) together with a selectable marker gene Amp^{r} was incorporated into an expression vector pET3d (manufactured by STRATAGENE) to obtain the plasmid pET-PGP113. In this plasmid, a glucan phosphorylase gene was operably linked under control of an isopropyl-β-D-thiogalactopyranoside (IPTG) inducible promoter. This plasmid was introduced into Escherichia coli BL21 (DE3) (manufactured by STRATAGENE) by the competent cell method. This Escherichia coli was plated on a plate containing LB medium (1% tryptone (manufactured by Difco), 0.5% yeast extract (manufactured by Difco), 1% sodium chloride, 1.5% agar)) containing the antibiotic ampicillin, and this was cultured at 37° C. overnight. Escherichia coli grown on this plate was selected to obtain Escherichia coli in which a potato-derived α-1,4-glucan phosphorylase gene had been introduced. It was confirmed by analyzing the sequence of the introduced gene that the resulting Escherichia coli contains the glucan phosphorylase gene. In addition, it was confirmed by activity measurement that the resulting Escherichia coli expresses the α-1,4-glucan phosphorylase.

This Escherichia coli was inoculated in 1 liter of LB medium (1% tryptone (manufactured by Difco), 0.5% yeast extract (manufactured by Difco), 1% sodium chloride) containing the antibiotic ampicillin, and this was cultured at 37° C. for 3 hours with shaking at 120 rpm. Thereafter, IPTG was added to this medium to 0.1 mM, and pyridoxine was added to this medium to 1 mM, and this was cultured with shaking at 22° C. for a further 20 hours. Then, this culture was centrifuged at 5,000 rpm for 5 minutes to collect the Escherichia coli cells. The resulting cells were suspended in 50 ml of 20 mM Tris-HCl buffer (pH 7.0) containing 0.05% Triton X-100 and, then, this was crushed by sonication to obtain 50 ml of a crushed cell liquid. This crushed liquid contained 4.7 U/mg glucan phosphorylase.

This crushed cell liquid was heated at 55° C. for 30 minutes. After heating, this was centrifuged at 8,500 rpm for 20 minutes to remove insoluble proteins and the like, to obtain the supernatant. The resulting supernatant (containing 125 mg of proteins) was applied to an anion exchange resin Q-Sepharose which had been pre-equilibrated using an equilibrating buffer (20 mM phosphate buffer, pH 7.0), allowing glucan phosphorylase to be adsorbed onto the resin. The resin was washed with a buffer containing 200 mM sodium chloride to remove impurities. Subsequently, the protein was eluted with a buffer containing 300 mM sodium chloride, to obtain the recombinant glucan phosphorylase enzyme solution.

### EXAMPLES 1-1 TO 1-6

### Synthesis of Amylose at Various Concentrations of Primer

Using reaction mixtures having the compositions (at reaction initiation) shown in the following Table 1, incubation was performed at 45° C. over 16 hours to synthesize amylose. amylose is desired to be synthesized, the primer may be used at a smaller amount and, when low molecular weight amylose is desired to be synthesized, the primer may be used at a larger amount.

### EXAMPLES 2-1 TO 2-5

### Synthesis of Amylose at Various Concentrations of Cellobiose Phosphorylase

Using reaction mixtures having the compositions (at reaction initiation) shown in the following Table 2, incubation was performed at 45° C. over 16 hours to synthesize amylose.

TABLE 2

| | Composition | | | | | Weight | |
|---|---|---|---|---|---|---|---|
| | Concentration | Concentration | Concentration | Concentration | Concentration | average | |
| | of CBP | of cellobiose | of phosphoric | of GP (U/g | of primer (G4*¹) | molecular | Yield |
| No. | (U/g cellobiose) | (%) | acid*² (mM) | cellobiose) | (μM) | weight | (%) |
| Example | 0.83 | 3 | 30 | 50 | 75 | 83,600 | 14.7 |
| 2-1 | | | | | | | |
| Example | 1.65 | 3 | 30 | 50 | 75 | 91,080 | 20.7 |
| 2-2 | | | | | | | |
| Example | 3.30 | 3 | 30 | 50 | 75 | 111,200 | 25.7 |
| 2-3 | | | | | | | |
| Example | 6.60 | 3 | 30 | 50 | 75 | 129,900 | 33.8 |
| 2-4 | | | | | | | |
| Example | 13.20 | 3 | 30 | 50 | 75 | 144,900 | 35.2 |
| 2-5 | | | | | | | |

*¹G4: maltotetraose
*²Phosphoric acid was added as a potassium dihydrogen phosphate-disodium hydrogen phosphate buffer. The pH of the phosphate buffer is 7.0.

After reaction, the weight average molecular weights and the yields of synthesized amyloses were determined according to the aforementioned 1.3 and 1.4. Results are shown in Table 2 and FIG. 3.

As a result, it was found that, up to 6.60 U/g cellobiose, as the amount of cellobiose phosphorylase is increased, the yield of amylose is increased, but when the concentration exceeds 6.60 U/g cellobiose, even if the amount of cellobiose phosphorylase is increased, the increase in yield of resulting amylose lessens. Therefore, it was found that the preferable concentration of the cellobiose phosphorylase is the concentration of 6.60 U/g cellobiose. In addition, since the yield of the amylose synthesizing reaction is 33.8% at maximum, it was confirmed, from these results, that production of amylose at an industrial level is possible.

### EXAMPLES 3-1 TO 3-5

### Synthesis of Amylose at Various Concentrations of Phosphoric Acid

Using reaction mixtures having the compositions (at reaction initiation) shown in the following Table 3, incubation were performed at 45° C. over 16 hours to synthesize amylose.

TABLE 3

| | Composition | | | | | Weight | |
|---|---|---|---|---|---|---|---|
| | Concentration | Concentration | Concentration | Concentration | Concentration | average | |
| | of phosphoric | of primer (G4*¹) | of cellobiose | of GBP (U/g | of GP (U/g | molecular | Yield |
| No. | acid*² (mM) | (μM) | (%) | cellobiose) | cellobiose) | weight | (%) |
| Example | 5 | 75 | 3 | 6.6 | 50 | 112,300 | 29.4 |
| 3-1 | | | | | | | |
| Example | 15 | 75 | 3 | 6.6 | 50 | 110,200 | 32.2 |
| 3-2 | | | | | | | |
| Example | 30 | 75 | 3 | 6.6 | 50 | 111,000 | 32.3 |
| 3-3 | | | | | | | |
| Example | 45 | 75 | 3 | 6.6 | 50 | 91,360 | 24.6 |
| 3-4 | | | | | | | |
| Example | 100 | 75 | 3 | 6.6 | 50 | 88,060 | 12.8 |
| 3-5 | | | | | | | |

*¹G4: maltotetraose
*²Phosphoric acid was added as a potassium dihydrogen phosphate-disodium hydrogen phosphate buffer. The pH of the phosphoric acid buffer is 7.0.

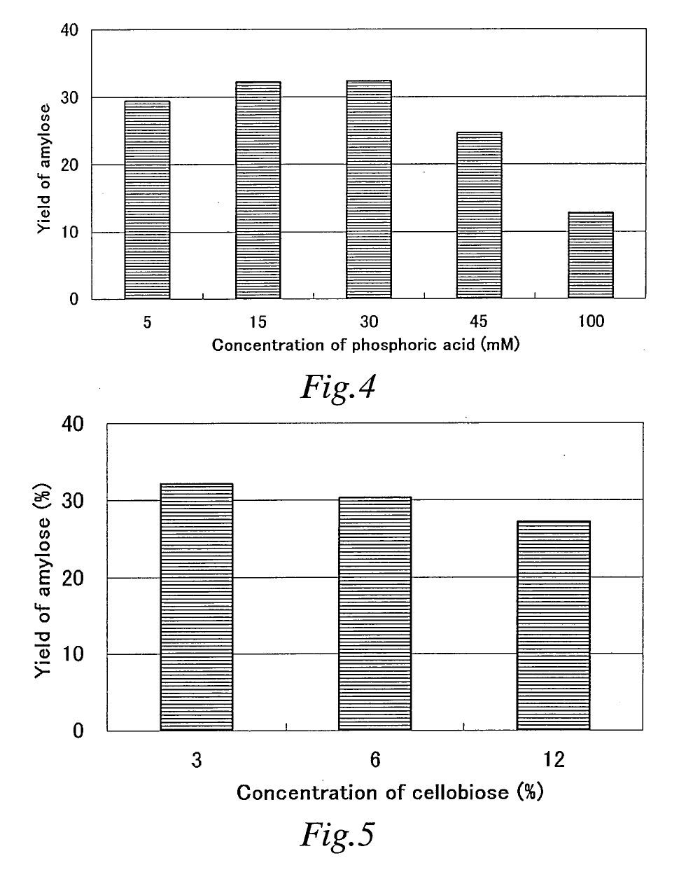

After reaction, the weight average molecular weights and yields of synthesized amyloses were determined according to the aforementioned 1.3 and 1.4. Results are shown in Table 3 and FIG. 4.

As a result, it was found that the yield of amylose is highest at a concentration of phosphoric acid of 15 mM to 30 mM, but since the yield of amylose is not considerably changed in the range of 5 mM to 45 mM, amylose can be synthesized efficiently in the range of 5 mM to 45 mM.

### EXAMPLES 4-1 TO 4-3

### Synthesis of Amylose at Various Concentrations of Cellobiose

Using reaction mixtures having the compositions (at reaction initiation) shown in the following Table 4, incubation were performed at 45° C. over 16 hours to synthesize amylose.

TABLE 4

| | Composition | | | | | Weight | |
|---|---|---|---|---|---|---|---|
| | Concentration | Concentration | Concentration | Concentration | Concentration | average | |
| | of cellobiose | of primer (G4*¹) | of phosphoric | of GBP (U/g | of GP (U/g | molecular | Yield |
| No. | (%) | (μM) | acid*² (mM) | cellobiose) | cellobiose) | weight | (%) |
| Example | 3 | 75 | 30 | 6.6 | 50 | 122,000 | 32.1 |
| 4-1 | | | | | | | |
| Example | 6 | 150 | 60 | 6.6 | 50 | 110,200 | 30.3 |
| 4-2 | | | | | | | |
| Example | 12 | 300 | 120 | 6.6 | 50 | 112,500 | 27.2 |
| 4-3 | | | | | | | |

*¹G4: maltotetraose
*²Phosphoric acid was added as a potassium dihydrogen phosphate-disodium hydrogen phosphate buffer. The pH of the phosphate buffer is 7.0.

After reaction, the weight average molecular weights and yields of synthesized amyloses were determined according to the aforementioned 1.3 and 1.4. Results are shown in Table 4 and FIG. 5.

As a result, when the concentration of cellobiose was increased without changing the concentration ratio of cellobiose, the primer and phosphoric acid, inhibition of amylose synthesis due to an increase in the concentration of cellobiose was not generated. Therefore, it was found that, in order to synthesize amylose in a large amount, the concentration of cellobiose can be increased.

### EXAMPLES 5-1 TO 5-4

### Synthesis of Amylose Using Glucose Isomerase, or Glucose Oxidase, Mutarotase and Peroxidase

Using reaction mixtures having the compositions (at reaction initiation) shown in the following Table 5, incubation was performed at 45° C. over 16 hours to synthesize amylose.

TABLE 5

| | Composition | | | | | |
|---|---|---|---|---|---|---|
| | Concentration | Concentration | | | | |
| | of | of | Concentration | Concentration | | |
| | glucose | glucose | of | of | | Concentration |
| | isomerase | oxidase | mutarotase | peroxidase | Concentration | of primer |
| Example | (U/g | (U/g | (U/g | (U/g | of cellobiose | (G4*¹) |
| No. | cellobiose) | cellobiose) | cellobiose) | cellobiose) | (%) | (μM) |
| 5-1 | 0 | 0 | 0 | 0 | 3 | 75 |
| 5-2 | 2.7 | 0 | 0 | 0 | 3 | 75 |
| 5-3 | 0 | 20 | 0.43 | 4.2 | 3 | 75 |
| 5-4 | 0 | 200 | 4.3 | 42 | 3 | 75 |

| | Composition | | | | |
|---|---|---|---|---|---|
| | Concentration | | | | |
| | of | Concentration | Concentration | Weight | |
| | phosphoric | of | of | average | |
| Example | acid*² | GBP (U/g | GP (U/g | molecular | Yield |
| No. | (mM) | cellobiose) | cellobiose) | weight | (%) |
| 5-1 | 30 | 6.6 | 50 | 136,100 | 32.8 |
| 5-2 | 30 | 6.6 | 50 | 169,100 | 45.6 |
| 5-3 | 30 | 6.6 | 50 | 132,700 | 54.9 |
| 5-4 | 30 | 6.6 | 50 | 133,400 | 64.8 |

*¹G4: maltotetraose
*²Phosphoric acid was added as a potassium dihydrogen phosphate-disodium hydrogen phosphate buffer. The pH of the phosphate buffer is 7.0.

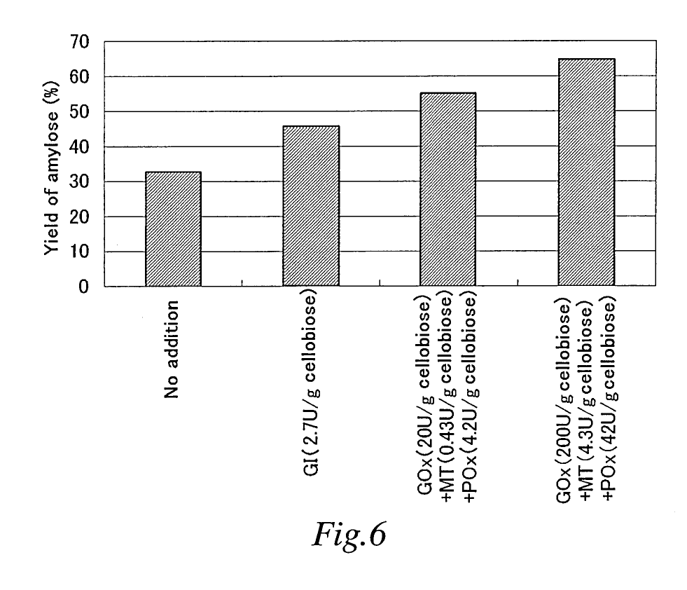

After reaction, the weight average molecular weights and yields of synthesized amylose were determined according to the aforementioned 1.3 and 1.4. Results are shown in Table 5 and FIG. 6.

As a result, it was found that the yield of amylose is dramatically improved by adding glucose isomerase (GI) or glucose oxidase (GOx)+mutarotase (MT)+peroxidase (POx) to the reaction system. Particularly, when glucose oxidase (GOx)+mutarotase (MT)+peroxidase (POx) were added, the yield of amylose was 64.8%, which is about 2-fold the yield (32.8%) of the case where these enzymes were not added.

It is thought that this improvement in the yield is due to the fact that since glucose produced by phosphorolysis of cellobiose inhibits the reaction of CBP and GP, the problem of reaction inhibition on CBP and GP can be avoided by degrading glucose in the reaction solution by GI or GOx to decrease its relative concentration.

### EXAMPLE 6

### Synthesis of Glucan Containing α-1,6 Branch

0.3 g of cellobiose and 0.75 micromoles of primer (G4) were dissolved in 10 ml of 30 mM phosphoric acid buffer (pH 7.0), then, 1.98 U of recombinant cellobiose phosphorylase obtained according to the aforementioned 2.1 preparation method, 15 U of recombinant potato α-1,4-glucan phosphorylase obtained according to the aforementioned 2.2 preparation method, and 1,500 U of Aquifex aeolicus derived branching enzyme prepared according to the method described in Example 1 of Japanese Patent Laid-Open Publication No. 2000-316581 were added thereto to prepare a reaction solution, and this reaction solution was incubated at45° C. for 16 hours. After completion of incubation, an equivalent volume of ethanol was added to the reaction solution to precipitate the glucan. Centrifugation was performed to recover precipitates, and the precipitates were lyophilized to obtain 0. 048 g of a glucan having a branched structure (yield about 32%).

(Analysis of Glucan Obtained in Example 6)

Whether the glucan synthesized in Example 6 has a branched structure or not, and the average unit chain length of the synthesized glucan were determined according to the methods described by H. Takata et al., Carbohydr. Res, 295, 91-101(1996). As a result, it was confirmed that the synthesized glucan has a branched structure, and the average unit chain length is 11. In this manner, it was found that, by further containing a branching enzyme in addition to CBP and GP in the reaction solution, a glucan having a branched structure can be synthesized from cellobiose.

### EXAMPLE 7

### Synthesis of Glucan Having Cyclic Structure

0.3 g of cellobiose and 0.75 micromoles of primer (G4) were dissolved in 10 ml of 30 mM phosphoric acid buffer (pH 7.0), then, 1.98 U of recombinant cellobiose phosphorylase obtained according to the aforementioned 2.1 preparation method, 15 U of recombinant potato α-1,4-glucan phosphorylase obtained according to the aforementioned 2.2 preparation method, and 1.5 U of Thermus aquaticus derived 4-α-glucanotransferase were added thereto to prepare a reaction solution, and this reaction solution was incubated at 45° C. for 16 hours. It should be noted that the Thermus aquaticus derived 4-α-glucanotransferase was prepared employing the only known DNA sequence of Thermus aquaticus 4-α-glucanotransferase by the same method as the method for α-1,4-glucan phosphorylase in the aforementioned 2.2.

After completion of incubation, an equivalent volume of ethanol was added to the reaction solution to precipitate the glucan. Centrifugation was performed, and precipitates were recovered, and the precipitates were lyophilized to obtain 0.05 g of a mixture of glucans having a cyclic structure (cyclic glucan) and a linear glucan (amylose) (yield about 33%).

(Analysis of Glucan Obtained in Example 7)

When 4-α-glucanotransferase acts on amylose, a cyclic glucan is excised and synthesized completely from amylose, and amylose which is shorter by the chain length of the cyclic glucan remains. Thus, the amount of the synthesized cyclic glucan was measured according to the method described in T. Takaha, M. Yanase, H. Takata, S. Okada and S. M. Sumith: J. Biol. Chem., 271,2902-2908(1996). In this method, amyloses in the solution are degraded into glucose units, and the amount of the remaining cyclic glucan is measured. As a result of this measurement, it was confirmed that a cyclic glucan was formed. In addition, when the yield of the cyclic glucan was calculated by comparing the amount of the measured cyclic glucan and the amount of the starting raw material cellobiose, it was found to be 9.6%. Therefore, it was found that about 29% of the glucan obtained in Example 7 is cyclic glucan, and the remaining about 71% is linear amylose. In this manner, it was found that, by further containing 4-α-glucanotransferase in addition to CBP and GP in the reaction solution, glucan having a cyclic structure can be synthesized from cellobiose.

### REFERENCE EXAMPLE 1

### Yield at Equilibrium for Sucrose Phosphorylase

In order to investigate the yield at equilibrium for sucrose phosphorylase (SP), the yield at equilibrium when G-1-P is a starting raw material was obtained.

Firstly,

G-1-P having a final concentration of 50 mM;

an enzyme (SP) having a final concentration of 50 U/ml;

an acceptor (fructose) having a final concentration of 50 mM; and

Tris-HCl (pH 7.0) having a final concentration of 50 mM were mixed, this was incubated at 45° C. for 6 hours or 16 hours, and the concentration of free phosphorus was measured by the molybdenum method. From the resulting concentration of phosphorus, the yield at equilibrium for this enzyme was obtained according to the following equation:
Yield at equilibrium (%)=concentration of phosphorus (mM)/50×100

Results are shown in the following Table 6:

TABLE 6

yield at Equilibrium
(%)

| Enzyme | Acceptor (50 mM) | 6 hours | 16 hours |
|---|---|---|---|
| Sucrose | Fructose (Fru) | 15.8 | 15.8 |
| phosphorylase | | | |

### REFERENCE EXAMPLE 2

### Yield of Product When Two Phosphorylases are Coupled in the Presence of Phosphoric Acid

Reaction yields in the case of the following two cases were obtained:
(2-1) Production of sucrose from cellobiose (CBP+SP+Fru);
(2-2) Production of sucrose from cellobiose in the presence of GOx+MT+POx (CBP+SP+Fru+GOx+MT+POx)

Firstly, starting raw material (cellobiose) having a final concentration of 50 mM, phosphoric acid buffer (pH 7.0) having a final concentration of 10, 30 or 100 mM, and each enzyme having a final concentration of 50 U/ml were mixed and reacted at 45° C. for 16 hours. After completion of the reaction, the reaction solution was degraded with invertase, and the concentration of free glucose was measured to obtain the concentration of sucrose. In both of these two reaction systems, sucrose is the product. From the resulting concentration of sucrose, the yield at equilibrium regarding each reaction system was obtained according to the following equation:
Yield at equilibrium (%)=concentration of sucrose (mM)/(50 m)×100

Results are shown in the following Table 7:

TABLE 7

| | | Without | With |
|---|---|---|---|
| | Pi | GOx + MT + POx | GOx + MT + POx |
| | concentration*¹ | Sucrose yield | Sucrose yield |
| Reaction | (mM) | (%) | (%) |
| Cellobiose | 10 | 1.22 | — |
| ↓ | 30 | 1.00 | 1.20 |
| sucrose | 100 | 1.50 | — |

*¹Phosphoric acid was added as a potassium dihydrogen phosphate-disodium hydrogen phosphate buffer. The pH of the phosphate buffer is 7.0.

As a result, the yield of the reaction synthesizing sucrose from cellobiose was extremely low even when the concentration of phosphoric acid was changed. In addition, increasing the yield of sucrose was attempted by eliminating glucose from the reaction system using glucose oxidase, mutarotase and peroxidase, but the yield was hardly increased.

### INDUSTRIAL APPLICABILITY

According to the method of the present invention, non-digestive β-1,4-glucan (particularly cellulose and partial degraded products thereof) can be converted into an edible food. Since β-1,4-glucan, which is a biomass present on the earth at a large amount can be converted into α-1,4-glucan at a low cost and high efficiency, the present invention also greatly contributes to solution of the food crisis problem, and the waste problem.

As described above, the present invention has been exemplified using a preferred embodiment of the present invention, but the present invention should not be construed to be limited to this embodiment. It is understood that the present invention should be construed for its scope only by the claims. It is understood that those skilled in the art can practice an equivalent range based on the description of the invention and the technical common knowledge, from the description of the specific preferable embodiment of the present invention. It is understood that patents, patent applications and publications cited in the present specification are herein incorporated by reference for the content thereof as if the contents themselves were specifically described in the present specification.

1. A method for production of an α-glucan from a β-1,4-glucan, comprising:
reacting a solution containing a β-1,4-glucan, a primer, a source of phosphoric acid, β-1,4-glucan phosphorylase, and α-1,4-glucan phosphorylase to produce an α-glucan.

2. The method according to claim 1, wherein said β-1,4-glucan is cellobiose, and said β-1,4-glucan phosphorylase is cellobiose phosphorylase.

3. The method according claim 1, wherein said β-1,4-glucan is a cellooligosacccharide having a degree of polymerization of 3 or more, and said β-1,4-glucan phosphorylase is cellodextrin phosphorylase.

4. The method according to claim 1, wherein said β-1,4-glucan is a cellooligosaccharide having a degree of polymerization of 3 or more, and said β-1,4-glucan phosphorylase is cellobiose phosphorylase and cellodextrin phosphorylase.

5. The method according to claim 1, wherein said production step further comprises removing glucose produced as a byproduct from said solution simultaneously with production of said α-glucan.

6. The method according to claim 5, wherein said solution further contains glucose isomerase or glucose oxidase.

7. The method according to claim 5, wherein said solution further contains glucose oxidase and mutarotase.

8. The method according to claim 7, wherein said solution further contains catalase or peroxidase.

9. The method according to claim 1, wherein said source of phosphoric acid is inorganic phosphoric acid, glucose-1-phosphate, or a mixture of inorganic phosphoric acid and glucose-1-phosphate.

10. The method according to claim 1, wherein the concentration of said source of phosphoric acid is 1 mM to 50 mM.

11. The method according to claim 1, wherein said α-glucan is amylose.
